(19) United States
(12) Patent Application Publication
(10) Pub. No.: US 2012/0309705 A1
(43) Pub. Date: Dec. 6, 2012

(54) NOVEL 3'-DEOXY-3'-METHYLIDENE-BETA-L-NUCLEOSIDES

(75) Inventors: Xiao Xiong Zhou, Huddinge (SE); Staffan Torssell, Bromma (SE); Olov Wallner, Ekero (SE); Piaoyang Sun, Huddinge (SE)

(73) Assignee: NOVADEX PHARMACEUTICALS AB, Huddinge (SE)

(21) Appl. No.: 13/513,727

(22) PCT Filed: Dec. 14, 2010
(86) PCT No.: PCT/SE2010/051374

§ 371 Date: Aug. 7, 2012

Related U.S. Application Data
(60) Provisional application No. 61/287,397, filed on Dec. 17, 2009.

(30) Foreign Application Priority Data
Dec. 17, 2009 (SE) 0950975-3

(51) Int. Cl. A61K 31/7072; A61K 31/7068; C07H 19/16; A61K 31/7076; A61P 31/18; C07D 473/00; C07D 473/30; C07F 9/6512; C07F 9/6561; C07H 19/06; C07D 239/22

(52) U.S. Cl. 514/45; 536/28.2; 514/50; 514/49; 536/28.53; 536/28.5; 536/27.6; 536/27.81; 544/318; 544/264; 544/314; 544/267; 544/243; 544/244

(57) ABSTRACT

The present invention includes novel 3′-deoxy-3′-methylidene-β-L-nucleosides, pharmaceutical composition comprising such compounds, as well as the methods to treat or to prevent viral infections and in particular HBV and/or HIV infections. In accordance with the present invention, there are provided compounds represented by Formula (I), wherein B is selected from A1 and A2;

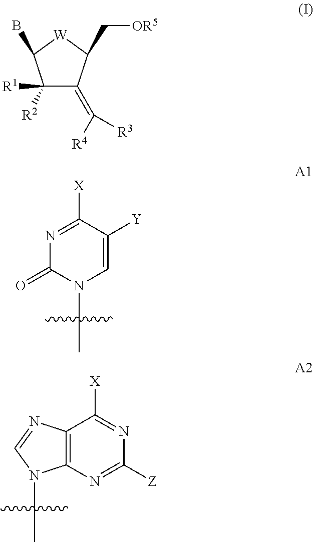

### FIELD OF THE INVENTION

The present invention relates to 3′-deoxy-3′-methylidene-β-L-nucleosides and their use for the treatment and prevention of viral infections in general and preferably HBV and/or HIV infections.

### BACKGROUND

Hepatitis B virus (HBV) is a DNA virus, and belongs to the family of hepadnaviridae. HBV is the causative agent for human hepatitis. It is estimated that more than 2 billion people have been infected with HBV at some stage in their lives and today there are some 300 million remaining chronically infected. HBV is transmitted through percutaneous or parenteral contact with infected blood, body fluid and by sexual intercourse. Another major route is perinatal transmission from mother to baby via blood or milk. The millions of HBV carriers are the constant source for the transfection of the virus. A significant portion of the HBV infected will develop chronic hepatitis, which is characterized by chronic liver necroinflammation and may lead to the progressive fibrosis. HBV is a major cause of human liver cancer. The mechanism by which HBV induces cancer is yet to be confirmed. It is postulated that HBV infection may directly trigger tumor development, or indirectly trigger tumor development through chronic inflammation, cirrhosis, and cell regeneration associated with the infection. HBV infection is the cause of up to 80% of all hepatocellular carcinoma worldwide and is also the major cause for liver failure. Overall, about 1 million patients die from HBV-related liver diseases each year.

HIV is another virus which imposes serious threats to human life. Human immunodeficiency virus (HIV) is a member of the retrovirus family, which causes AIDS. HIV primarily infects vital cells in the human immune system such as helper T cells (specifically CD4⁺ T cells), macrophages, and dendritic cells. HIV infection leads to low levels of CD4⁺ T cells through three main mechanisms: firstly, direct viral killing of infected cells; secondly, increased rates of apoptosis in infected cells; and thirdly, killing of infected CD4⁺ T cells by CD8 cytotoxic lymphocytes that recognize infected cells. When CD4⁺ T cell numbers decline below a critical level, cell-mediated immunity is lost, and the body becomes progressively more susceptible to opportunistic infections. Most people infected with HIV may eventually develop AIDS. These individuals may die from opportunistic infections or malignancies associated with the progressive failure of the immune system. HIV infection in humans is considered pandemic by the World Health Organization (WHO). An estimated 33 million people are living with HIV today, of whom several millions are children. Around 2.5 million new infections occurred each year. In 2007, 2.1 million people died of AIDS-related illnesses. The effective and safe anti-HBV and anti-HIV therapeutics are highly needed to treat and prevent the diseases and the infections.

Both HBV and HIV encode their own polymerases, which are responsible for the synthesis of viral genomes. The HBV polymerase (also called HBV reverse transcriptase) and HIV reverse transcriptase are multifunctional proteins, which have the reverse transcriptase activity, the DNA-dependent DNA polymerase activity and the RNase H activity. The enzymes are essential for the viral replication, and the blocking of their activity will abolish the viral replication completely. The HBV polymerase and HIV reverse transcriptase have been established as the attractive targets for the anti-viral therapies. Indeed, a substantial achievement has been made in developing the effective HBV and HIV polymerase inhibitors. The nucleoside/nucleotide polymerase inhibitors are an important class of viral polymerase inhibitors. They can be regarded as the prodrugs, and need the activation for their antiviral efficacy through a phosphorylation process to their nucleoside triphosphates or nucleotide diphosphates that function as the inhibitors for the viral polymerases.

In the last two decades, a number of nucleoside/nucleotide polymerase inhibitors have been developed for the treatment of HIV and HBV infections. Some important inhibitors for HIV infection include zidovudine, stavudine, didanosine lamivudine, emtricitabine, tenofovir and abacavir. For the treatment of HBV infection, there are lamivudine, adefovir, tenofovir, entecavir and telbivudine. Those inhibitors have provided the methods and means for treating HIV and HBV infection and have been proved and accepted as an indispensible part of the HIV and HBV therapy. However, many severe adverse effects have been found to be associated with the treatment using those nucleoside/nucleotide inhibitors, for example, bone marrow toxicity, lactic acidosis, myopathy, hepatomegaly with steatosis, nephrotoxicity, peripheral neuropathy, pancreatitis, lipodystrophy and so on. Another major problem associated with the nucleoside/nucleotide inhibitors is the development of resistance towards the therapies. For example, the HBV polymerase mutation of rtM204I (ATG to ATA) and rtM204V (ATG to GTG) reduces the susceptibility towards lamivudine by 550 and 153 folds, respectively (Allen, M. I. et al., Hepatol., 1998, 27, 1670-1677). Beside rtM204 mutations, rtL180M mutation was found to be common, which brings about a loss of sensitivity to lamivudine about 18 fold (Leung, N., J. Gastroenterol. Hepatol., 2000, 15, (suppl.), E53-E60). The double mutants containing rtL180M and rtM204V confer the activity loss of about thousand folds for lamivudine (Jarvis, B., and Fauld, D., Drugs, 1999, 58, 101-141). The mutants resistant to lamivudine were also found to have cross-resistance to entecavir, telbivudine. The mutant strains with rtN236T in HBV polymerase has been isolated, leading to the loss of susceptibility to adefovir about 10 fold. The mutation rtA181V was also found, which causes the activity loss of adefovir about 33 fold (Angus, P. et al., Gastroenter. 2003, 125, 292-297). For HIV, due to the high replication rate and the low fidelity of the HIV reverse transcriptase, the resistance has been the key issue in the HIV treatment. The mutants at various residues of HIV reverse transcriptase have been identified, for example, M41L, K65R, D67N, T69D, K70R, L74V, V75T, M184V, M184I, L210W, T215Y and K219E. Those mutants result in a substantially lower efficacy of the treatment and lead to the failure of the treatment.

In light of the fact that HIV infection and HBV infection have reached epidemic levels worldwide, and have tragic effects on the infected patients, there remains a strong need to provide new, effective and safe pharmaceutical agents to treat these diseases and particularly the new therapeutics which are effective in treating the HBV and HIV infections that are resistant to the current therapeutics.

Therefore, it is an object of the present invention to provide novel compounds, methods and compositions for the treatment of human patients infected by viruses, particularly with HBV or HIV.

### DISCLOSURE OF THE INVENTION

The present invention includes novel 3′-deoxy-3′-methylidene-β-L-nucleosides, pharmaceutical compositions comprising such compounds, as well as methods to treat or to prevent viral infections and in particular HBV and/or HIV infections. In accordance with the present invention, there are provided compounds represented by the Formula (I).

Thus, in one aspect of the invention, there are provided compounds of the general Formula (I)

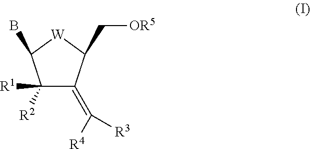

wherein
B is selected from A1 and A2;

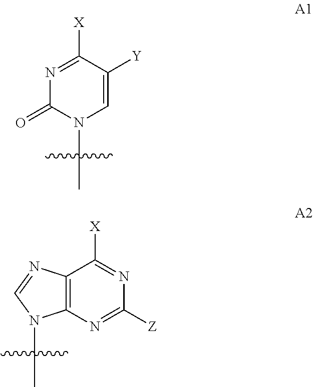

X is selected from H, OH, NH₂, halogen, (C₁-C₆alkyl)NH and (C₃-C₆cycloalkyl)NH;
Y is selected from H, halogen, C₂-C₆alkenyl and C₁-C₃alkyl;
Z is selected from H, halogen and NH₂;
W is selected from O, S and CH₂;
R¹ and R² are independently selected from H, F, OH, OCH₃ and CH₃;
R³ and R⁴ are independently selected from H, F and CH₃;
R⁵ is selected from H, phosphate, diphosphate and triphosphate;
or a pharmaceutically acceptable salt or prodrug thereof.

In another aspect of the invention there are provided compounds of the general Formula (I)

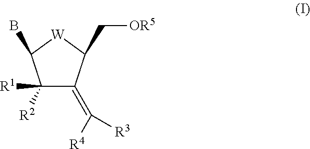

wherein
B is selected from A1 and A2;

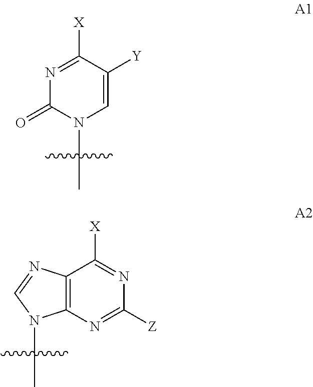

X is selected from H, OH, NH₂, halogen, (C₁-C₆alkyl)NH and (C₃-C₆cycloalkyl)NH;
Y is selected from H, halogen, C₂-C₆alkenyl and C₁-C₃alkyl;
Z is selected from H, halogen and NH₂;
W is selected from O, S and CH₂;
R¹ and R² are independently selected from H, F, OH, OCH₃ and CH₃;

R³ and R⁴ are independently selected from H, F and CH₃;

R⁵ is selected from H, phosphate, diphosphate and triphosphate;

provided that when W is O; R¹ is H; and R² is OH, F or OCH₃, then R³ and R⁴ are not both F; or R³ and R⁴ are not both H; and

provided that when W is O; R² is H; and R¹ is OH, OCH₃ or F, then R³ and R⁴ are not both F; or R³ and R⁴ are not both H;
or a pharmaceutically acceptable salt or prodrug thereof.

In another aspect of the invention there are provided compounds of the general Formula (I), wherein W is O.

In another aspect of the invention there are provided compounds of the general Formula (I), wherein W is S or CH₂.

In another aspect of the invention there are provided compounds of the general Formula (I), wherein R¹ and R² is H.

In another aspect of the invention there are provided compounds of the general Formula (I), wherein R³ and R⁴ are independently selected from F and CH₃; provided that R³ and R⁴ are not both F.

In another aspect of the invention there are provided compounds of the general Formula (I), wherein R³ is H; R⁴ is selected from F and CH₃.

In another aspect of the invention there are provided compounds of the general Formula (I), wherein R⁴ is H; R³ is selected from F and CH₃.

In another aspect of the invention there are provided compounds of the general Formula (I), wherein R¹ is CH₃.

In another aspect of the invention there are provided compounds of the general Formula (I), wherein R² is CH₃.

In another aspect of the invention there are provided compounds of the general Formula (I), wherein R¹, R², R³ and R⁴ are H.

In another aspect of the invention there are provided compounds of the general Formula (I), wherein X is selected from H, OH, and NH₂; Y is selected from H, F and CH₃; and Z is selected from H and NH₂.

In another aspect of the invention there are provided compounds of the general Formula (I), wherein W is O; R² is OH or OCH₃; and R¹, R³ and R⁴ are H.

In another aspect of the invention there are provided compounds of the general Formula (I), wherein W is O; R² is F; and R³ and R⁴ are H.

In another aspect of the invention there are provided compounds of the general Formula (I), wherein W is O; R² is CH₃; and R¹, R³ and R⁴ are H.

In another aspect of the invention there are provided compounds of the general Formula (I), wherein W is O; R¹ is F; and R², R³ and R⁴ are H.

In another aspect of the invention there are provided compounds of the general Formula (I), wherein W is O; R¹ is OH or OCH₃; and R², R³ and R⁴ are H.

In another aspect of the invention there are provided compounds of the general Formula (I), wherein B is Al; X is NH₂ or OH; Y is H, F or CH₃; W is O; R¹, R², R³ and R⁴ are H.

In another aspect of the invention there are provided compounds of the general Formula (I), wherein B is A2; X is NH₂, OH or H; Z is H or NH₂; W is O; R¹, R², R³ and R⁴ are H.

In another aspect of the invention there are provided compounds of the general Formula (I), wherein X is OH.

In another aspect of the invention there are provided compounds of the general Formula (I), wherein X is NH₂.

In another aspect of the invention there are provided compounds of the general Formula (I), wherein Y is F.

In another aspect of the invention there are provided compounds of the general Formula (I), wherein R⁵ is H.

In another aspect of the invention there are provided compounds of the general Formula (I), wherein R⁵ is phosphate, diphosphate or triphosphate.

In another aspect of the invention there are provided compounds of the general Formula (I), selected from:
1-(2,3-dideoxy-3-methylidene-β-L-pentofuranosyl)uracil;
1-(2,3-dideoxy-3-methylidene-β-L-pentofuranosyl)cytosine;
1-(3-deoxy-3-methylidene-β-L-pentoribofuranosyl)uracil;
1-(3-deoxy-3-methylidene-β-L-pentoribofuranosyl)cytosine;
1-(3-deoxy-3-methylidene-β-L-arabinopentofuranosyl)uracil;
1-(3-deoxy-3-methylidene-β-L-arabinopentofuranosyl)cytosine;
1-(2,3-dideoxy-3-methylidene-β-L-pentofuranosyl)thymine; and
9-(2,3-dideoxy-3-methylidene-β-L-pentofuranosyl)guanine;
   or a pharmaceutically acceptable salt or prodrug thereof.

In another aspect of the invention there are provided compounds of the general Formula (I), selected from:
1-(2,3-dideoxy-3-methylidene-β-L-pentofuranosyl)cytosine;
1-(3-deoxy-3-methylidene-β-L-pentoribofuranosyl)cytosine;
1-(3-deoxy-3-methylidene-β-L-arabinopentofuranosyl)cytosine;
1-(2,3-dideoxy-3-methylidene-β-L-pentofuranosyl)thymine; and
9-(2,3-dideoxy-3-methylidene-β-L-pentofuranosyl)guanine;
   or a pharmaceutically acceptable salt or prodrug thereof.

In another aspect of the invention there are provided compounds of the general Formula (I), selected from:
1-[2-deoxy-2-(S)-fluoro-3-deoxy-3-methylidene-β-L-pentofuranosyl]uracil;
1-[2-deoxy-2-(S)-fluoro-3-deoxy-3-methylidene-β-L-pentofuranosyl]cytosine;
1-[2-deoxy-2-(R)-fluoro-3-deoxy-3-methylidene-β-L-pentofuranosyl]uracil;
1-[2-deoxy-2-(R)-fluoro-3-deoxy-3-methylidene-β-L-pentofuranosyl]cytosine;
1-[(2S,3S,5R)-5-(Hydroxymethyl)-3-methyl-4-methylenetetrahydrofuran-2-yl]pyrimidine-2,4(1H,3H)-dione;
4-Amino-1-[(2S,3S,5R)-5-(hydroxymethyl)-3-methyl-4-methylenetetrahydrofuran-2-yl]pyrimidin-2(1H)-one;
1-(2,3-dideoxyl-3-methylidene-β-L-pentofuranosyl)-5-fluorouracil;
1-(2,3-dideoxyl-3-methylidene-β-L-pentofuranosyl)-5-fluorocytosine; and
9-(2,3-dideoxyl-3-methylidene-β-L-pentofuranosyl)adenine;
   or a pharmaceutically acceptable salt or prodrug thereof.

In another aspect of the invention there is provided a pharmaceutical composition for the treatment or prevention of a DNA virus infection and/or a retroviral infection in a host comprising an effective amount of a compound of the general Formula (I).

In another aspect of the invention there is provided a pharmaceutical composition for the treatment or prevention of HBV infections and/or HBV viruses which are resistant to one or more other anti-HBV drugs, comprising an effective amount of a compound of the general Formula (I).

In another aspect of the invention there is provided a pharmaceutical composition for the treatment or prevention of HIV infections and/or HIV viruses which are resistant to one or more other anti-HIV drugs, comprising an effective amount of a compound of the general Formula (I).

In another aspect of the invention there is provided a pharmaceutical composition, described above, which further comprises one or more additional agents having antiviral effects. Such agents may be anti-HIV agents, including the following non-limting examples: etravirine, efavirenz, delavirdine, nevirapine, lamivudine, zidovudine, emtricitabine, abacavir, tenofovir (or its prodrug), didanosine, stavudine, tipranavir, indinavir, saquinavir, lopinavir, ritonavir, amprenavir, fosamprenavir, darunavir, atazanavir, nelfinavir, maraviroc, enfuvirtide, raltegravir, vicriviroc, elvitegravir, bevirimat, racivir, apricitabine, elvucitabine, brecanavir, rilpivirine, SCH 532706, S/GSK1265744, IDX899 and GSK-364735. Such agents may also represent anti-HBV agents including the following non-limting examples: entecavir, lamivudine, adefovir (or its prodrug), telbivudine, tenofovir (or its prodrug), torcitabine, valtorcitabine, emtricitabine, clevudine, penciclovir (or famciclovir), interferon alfa-2b and peginterferon alfa-2a.

In another aspect of the invention there is provided a compound of the general Formula (I), for use in therapy.

In another aspect of the invention there is provided a compound of the general Formula (I), for use in the treatment or prevention of a DNA virus infection and/or retroviral infection.

In another aspect of the invention there is provided a compound of the general Formula (I), for use in the treatment or prevention of a HBV infection and/or a HBV virus which is resistant to one or more other anti-HBV drugs.

In another aspect of the invention there is provided a compound of the general Formula (I), for use in the treatment or prevention of a HIV infection and/or a HIV virus which is resistant to one or more other anti-HIV drugs.

In another aspect of the invention there is provided a compound of the general Formula (I), for use in the treatments or preventions as described above, which further comprises one or more additional agents having antiviral effects. Such agents may be anti-HIV agents, including the following non-limting examples: etravirine, efavirenz, delavirdine, nevirapine, lamivudine, zidovudine, emtricitabine, abacavir, tenofovir (or its prodrug), didanosine, stavudine, tipranavir, indinavir, saquinavir, lopinavir, ritonavir, amprenavir, fosamprenavir, darunavir, atazanavir, nelfinavir, maraviroc, enfuvirtide, raltegravir, vicriviroc, elvitegravir, bevirimat, racivir, apricitabine, elvucitabine, brecanavir, rilpivirine, SCH 532706, S/GSK1265744, IDX899 and GSK-364735. Such agents may also represent anti-HBV agents including the following non-limting examples: entecavir, lamivudine, adefovir (or its prodrug), telbivudine, tenofovir (or its prodrug), torcitabine, valtorcitabine, emtricitabine, clevudine, penciclovir (or famciclovir), interferon alfa-2b and peginterferon alfa-2a.

In another aspect of the invention there is provided use of a compound of the general Formula (I), in the manufacture of a medicament for treatment or prevention of a DNA virus infection and/or retroviral infection.

In another aspect of the invention there is provided use of a compound of the general Formula (I), in the manufacture of a medicament for treatment or prevention of a HBV virus infection; or a HBV virus, which is resistant to one or more other anti-HBV drugs.

In another aspect of the invention there is provided use of a compound of the general Formula (I), in the manufacture of a medicament for treatment or prevention of a HIV virus infection; or a HIV virus, which is resistant to one or more other anti-HIV drugs.

In another aspect of the invention there is provided use of a compound of the general Formula (I), in the manufacture of a medicament for treatments or preventions as described above, which further comprises one or more additional agents having antiviral effects. Such agents may be anti-HIV agents, including the following non-limting examples: etravirine, efavirenz, delavirdine, nevirapine, lamivudine, zidovudine, emtricitabine, abacavir, tenofovir (or its prodrug), didanosine, stavudine, tipranavir, indinavir, saquinavir, lopinavir, ritonavir, amprenavir, fosamprenavir, darunavir, atazanavir, nelfinavir, maraviroc, enfuvirtide, raltegravir, vicriviroc, elvitegravir, bevirimat, racivir, apricitabine, elvucitabine, brecanavir, rilpivirine, SCH 532706, S/GSK1265744, IDX899 and GSK-364735. Such agents may also represent anti-HBV agents including the following non-limting examples: entecavir, lamivudine, adefovir (or its prodrug), telbivudine, tenofovir (or its prodrug), torcitabine, valtorcitabine, emtricitabine, clevudine, penciclovir (or famciclovir), interferon alfa-2b and peginterferon alfa-2a.

In another aspect of the invention there is provided a method for the treatment or prevention of a DNA virus infection and/or retroviral infection in a subject in need thereof, comprising administering a therapeutically effective amount of a compound of the general Formula (I).

In another aspect of the invention there is provided a method for the treatment or prevention of a HBV infection; or a HBV virus wherein said HBV virus is resistant to one or more other anti-HBV drugs, in a subject in need thereof, comprising administering a therapeutically effective amount of a compound of the general Formula (I).

In another aspect of the invention there is provided a method for the treatment or prevention of a HIV infection; or a HIV virus wherein said HIV virus is resistant to one or more other anti-HIV drugs, in a subject in need thereof, comprising administering a therapeutically effective amount of a compound of the general Formula (I).

In another aspect of the invention there is provided a method as described above, which further comprises one or more additional agents having antiviral effects. Such agents may be anti-HIV agents, including the following non-limting examples: etravirine, efavirenz, delavirdine, nevirapine, lamivudine, zidovudine, emtricitabine, abacavir, tenofovir (or its prodrug), didanosine, stavudine, tipranavir, indinavir, saquinavir, lopinavir, ritonavir, amprenavir, fosamprenavir, darunavir, atazanavir, nelfinavir, maraviroc, enfuvirtide, raltegravir, vicriviroc, elvitegravir, bevirimat, racivir, apricitabine, elvucitabine, brecanavir, rilpivirine, SCH 532706, S/GSK1265744, IDX899 and GSK-364735. Such agents may also represent anti-HBV agents including the following non-limting examples: entecavir, lamivudine, adefovir (or its prodrug), telbivudine, tenofovir (or its prodrug), torcitabine, valtorcitabine, emtricitabine, clevudine, penciclovir (or famciclovir), interferon alfa-2b and peginterferon alfa-2a.

The invention further comprises the following compounds:
1-(2-deoxy-2-(R)—C-methyl-3-deoxy-3-methylidene-β-L-pentofuranosyl)cytosine;
1-(2-deoxy-2-(S)-fluoro-3-deoxy-3-methylidene-β-L-pentofuranosyl)thymine;
1-(2-deoxy-2-(R)-fluoro-3-deoxy-3-methylidene-β-L-pentofuranosyl)thymine;
1-(2-deoxy-2-(R)—C-methyl-3-deoxy-3-methylidene-β-L-pentofuranosyl)thymine;
1-(2-deoxy-2-(S)—C-methyl-3-deoxy-3-methylidene-β-L-pentofuranosyl)thymine;
1-(3-deoxy-3-methylidene-β-L-pentoribofuranosyl)thymine;
1-(3-deoxy-3-methylidene-β-L-arabinopentofuranosyl)thymine;
1-(2-deoxy-2-(S)-fluoro-3-deoxy-3-methylidene-β-L-pentofuranosyl)-5-fluorocytosine;
1-(2-deoxy-2-(R)-fluoro-3-deoxy-3-methylidene-β-L-pentofuranosyl)-5-fluorocytosine;
1-(2-deoxy-2-(R)—C-methyl-3-deoxy-3-methylidene-β-L-pentofuranosyl)-5-fluorocytosine;
1-(2-deoxy-2-(S)—C-methyl-3-deoxy-3-methylidene-β-L-pentofuranosyl)-5-fluorocytosine;
1-(3-deoxy-3-methylidene-β-L-pentoribofuranosyl)-5-fluorocytosine;
1-(3-deoxy-3-methylidene-β-L-arabinopentofuranosyl)-5-fluorocytosine;
9-(2-deoxy-2-(S)-fluoro-3-deoxy-3-methylidene-β-L-pentofuranosyl)guanine;
9-(2-deoxy-2-(R)-fluoro-3-deoxy-3-methylidene-β-L-pentofuranosyl)guanine;
9-(2-deoxy-2-(R)—C-methyl-3-deoxy-3-methylidene-β-L-pentofuranosyl)guanine;
9-(2-deoxy-2-(S)—C-methyl-3-deoxy-3-methylidene-β-L-pentofuranosyl)guanine;
9-(3-deoxy-3-methylidene-β-L-pentoribofuranosyl)guanine;
9-(3-deoxy-3-methylidene-β-L-arabinopentofuranosyl)guanine;
9-(3-deoxy-3-methylidene-β-L-pentoribofuranosyl)adenine;
9-(3-deoxy-3-methylidene-β-L-arabinopentofuranosyl)adenine;
9-(2-deoxy-2-(S)-fluoro-3-deoxy-3-methylidene-β-L-pentofuranosyl)adenine;
9-(2-deoxy-2-(R)-fluoro-3-deoxy-3-methylidene-β-L-pentofuranosyl)adenine;
9-(2-deoxy-2-(R)—C-methyl-3-deoxy-3-methylidene-β-L-pentofuranosyl)adenine; and
9-(2-deoxy-2-(S)—C-methyl-3-deoxy-3-methylidene-β-L-pentofuranosyl)adenine;
   or a pharmaceutically acceptable salt or prodrug thereof.

In another aspect of the invention, there is provided a method of treating and/or preventing HIV infections comprising the administration of a therapeutically effective amount of a compound of formula (I) or a pharmaceutically acceptable salt or prodrug thereof together with one or more of anti-HIV agents, for example, etravirine, efavirenz, delavirdine, nevirapine, lamivudine, zidovudine, emtricitabine, abacavir, tenofovir (or its prodrug), didanosine, stavudine, tipranavir, indinavir, saquinavir, lopinavir, ritonavir, amprenavir, fosamprenavir, darunavir, atazanavir, nelfinavir, maraviroc, enfuvirtide, raltegravir, vicriviroc, elvitegravir, bevirimat, racivir, apricitabine, elvucitabine, brecanavir, rilpivirine, SCH 532706, S/GSK1265744, IDX899 and GSK-364735.

In another aspect of the invention, there is provided a method of treating and/or preventing HBV infections comprising the administration of a therapeutically effective amount of a compound of formula (I) or a pharmaceutically acceptable salt thereof together with one or more of anti-HBV agents, for example entecavir, lamivudine, adefovir (or its prodrug), telbivudine, tenofovir (or its prodrug), torcitabine, valtorcitabine, emtricitabine, clevudine, penciclovir (or famciclovir), interferon alfa-2b and peginterferon alfa-2a.

The individual components of such combinations may be administered either sequentially or simultaneously in separate or combined pharmaceutical formulations.

Whenever used foregoing and hereinafter, the term ‘compounds of Formula (I)’, or ‘the compounds of the invention”, or “the compounds of the present invention” or similar terms, it is meant to include the compounds of Formula (I), their pharmaceutically acceptable prodrugs, salts, solvates, quaternary amines and metal complexes.

The term ‘prodrug’ as used throughout this text means the pharmacologically acceptable derivatives such as esters, carbamate, carbonate, ether, amides and phosphates, such that the resulting in vivo biotransformation product of the derivative is the active drug as defined in the compounds of Formula (I). The references describing prodrugs generally are hereby incorporated (Goodman and Gilman, The Pharmacological Basis of Therapeutics, 8^{th}ed, McGraw-Hill, Int. Ed. 1992, “Biotransformation of Drugs”, p 13-15; H. Bundgaard, Design of Prodrugs, H. Bundgaard ed. Elsevier Science Publisher, 1985; M. Taylor, Adv. Drug Delivery 1996, 19, 131; H. Bundgaard, Drugs of the Future, 1991, 16, 443; A. Simplicio, Molecules, 2008, 13, 519; P. Ettmayer, J. Med. Chem. 2004, 47, 2393). Particularly relevant are the prodrugs described for making nucleoside or nucleotide prodrugs (S. Hecker et al, J. Med. Chem., 2008, 51, 2328; P. Poijarvi-Virta et al, Current Med. Chem. 2006, 13, 3441; N. Gisch et al, J Med. Chem. 2008, 51, 6752; L. Wiebe et al, Adv. Drug Delivery Rev. 1999, 39, 63; J. Cooperwood et al, Nucleoside and Nucleotide Prodrugs, in Recent Advances in Nucleosides: Chemistry and Chemotherapy, C. K. Chu ed. Elsevier, 2002, p. 91-147), including the 5′-(O-arylphosphoramidate) prodrugs as described in the literature (D. Cahard et al Mini-Rev. Med. Chem., 2004, 4, 371; C. McGuigan et al, J. Med. Chem., 1996, 39, 1748; C. McGuigan et al, Antiviral Res., 1997, 35, 195; D. Saboulard et al, Mol. Pharmacol., 1999, 56, 693). Prodrugs preferably have excellent aqueous solubility, increased bioavailability and are readily metabolized into the compounds of Formula (I) in vivo. Prodrugs of a compound of the present invention may be prepared by modifying functional groups present in the compounds in such a way that the modifications are cleaved, either by routine manipulation or in vivo, to the parent compound.

Preferred are pharmaceutically acceptable ester, ether, carbonate, phosphoramidate or carbamate prodrugs that are hydrolysable in vivo and are derived from those compounds of Formula (I) having a hydroxy and/or an amino and/or a phosphate group. An in vivo hydrolysable ester, ether, carbonate, phosphoramidate or carbamate is an ester, ether, carbonate, phosphoramidate or carbamate, which is hydrolysed in the human or animal body to produce the parent alcohol, amine or phosphate. Suitable pharmaceutically acceptable esters for the hydroxyl of the compounds of the invention include, but not limited to, C₁-C₁₈alkanoyl ester, benzoyl ester, amino substituted carboxylic acid ester, hydroxyl substituted carboxylic acid esters, alkoxy substituted carboxylic acid esters, carboxyl substituted carboxylic acid esters. Some examples of such esters include, acetate, propanoate, butyrate, isobutyrate, pivalate, alanine ester, valine ester, isoleucine ester, lactate, malate, succinate and so on.

There is also provided pharmaceutically acceptable salts of the compounds of Formula (I) of the present invention. By the term “a pharmaceutically acceptable salt” is meant those derived from pharmaceutically acceptable inorganic and organic acids and bases. A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, nitric, methansulphonic, sulphuric, phosphoric, trifluoroacetic, para-toluene sulphonic, 2-mesitylen sulphonic, citric, acetic, tartaric, fumaric, lactic, succinic, malic, malonic, maleic, 1,2-ethanedisulphonic, adipic, aspartic, benzenesulphonic, benzoic, ethanesulphonic or nicotinic acid. In addition a suitable pharmaceutically acceptable salt of a compound of the invention, is, for example, a base-addition salt of a compound of the invention which is sufficiently acidic, for example, a metal salt, for example, sodium, potassium, calcium, magnesium, zinc or aluminum, an ammonium salt, a salt with an organic base which affords a physiologically acceptable cation, which includes quarternary ammonium ion, for example methylamine, ethylamine, diethylamine, trimethylamine, tert-butylamine, triethylamine, dibenzylamine, N,N-dibenzylethylamine, cyclohexylethylamine, tris-(2-hydroxyethyl)amine, hydroxyethyl diethylamine, (1R, 2S)-2-hydroxyinden-1-amine, morpholine, N-methylpiperidine, N-ethylpiperidine, piperazine, methylpiperazine, adamantylamine, choline hydroxide, tetrabutylammonium hydroxide, tris-(hydroxymethyl)methylamine hydroxide, L-arginine, N-methyl D-glucamine, lysine, arginine and the like.

Certain compounds of the present invention may exist as solvates or hydrates. It is to be understood that the present invention encompasses all such solvates or hydrates.

The compounds of the present invention may also contain unnatural proportions of atomic isotopes at one or more of the atoms that constitute such compounds. For example, the compounds may be radiolabeled with radioactive isotopes, such as for example tritium (³H), deuterium (²H), iodine-125 (¹²⁵I) or carbon-14 (¹⁴C). All isotopic variations of the compounds of the present invention, whether radioactive or not, are intended to be encompassed within the scope of the present invention.

Where tautomers exist in the compounds of the invention, we disclose all individual tautomeric forms and combinations of these as individual specific embodiments of the invention. For examples, nucleobases such as guanine, thymine and uacil may exist as an equilibrium of their keto and enol forms at their 6-position or 4-position respectively. It is understood that all individual tautomeric forms and combinations of these tautomers present in guanine, thymine and uracil are included in the present invention.

The compounds according to the invention have the core structures of β-L-nucleosides configuration, which have the defined stereochemistry at both 1′- and 4′-positions of the pentose ring. The present invention relates only to the β-L-nucleosides with the stereochemistry specified by Formula (I). However, the variables in the Formula (I), for example X and/or Y and the prodrugs of the compounds of Formula (I), may contain one or more asymmetrically substituted carbon atoms, asymmetric or chiral centres. The presence of one or more of these asymmetric centres in compounds according to the invention can give rise to stereochemically isomeric forms, stereoisomers. Unless the stereochemistry is clearly defined for example like β-L-nucleosides or by the chemical structures, in each case the invention is to be understood to possibly extend to all such stereoisomers, both in pure form and mixed with each others, including enantiomers and diastereomers, and mixtures including racemic mixtures thereof.

It will be appreciated that the compounds of formula (I) may have metal binding, chelating or complex forming properties and therefore may exist as metal complexes or metal chelates. Such metalated derivatives of the compounds of formula (I) are intended to be included within the scope of the invention.

The scientific and technological terms and nomenclatures used foregoing and hereinafter have the same meaning as commonly understood by a person of ordinary skill in the art, in addition, the following definitions shall apply throughout the specification and the appended claims unless specifically stated otherwise:

The term “halogen” denotes fluoro, chloro, bromo and iodo groups.

The term “C₁-C₃alkyl” denotes a straight or branched saturated alkyl group having 1 to 3 carbon atoms. Examples of said alkyls include methyl, ethyl, propyl and isopropyl. The term “C₁-C₆alkyl” denotes a straight or branched saturated alkyl group having 1 to 6 carbon atoms. Examples of said alkyls include, but are not limited to, methyl, ethyl, propyl, isopropyl, butyl, isobutyl, pentyl, isopentyl and hexyl.

The term “C₂-C₆alkenyl” denotes a straight or branched alkenyl group having saturated carbon-carbon bonds and at least one carbon-carbon double bond, and having 2 to 6 carbon atoms. Examples of said alkenyl include, but are not limited to, ethenyl, 1-propenyl, 2-propenyl, isopropenyl and butenyl.

The term “C₃-C₆cycloalkyl” denotes a saturated monocyclic ring having 3 to 6 carbon atoms. Examples of said cycloalkyl include, but are not limited to cyclopropyl, cyclobutyl, cyclopentyl and cyclohexyl.

The terms “phosphate”, “diphosphate” and “triphosphate” denote the following structures and their salts:

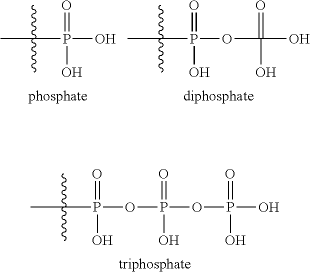

Unless otherwise indicated, the alkyl, alkenyl, alkoxy and cycloalkyl, (such as in C₁-C₆alkyl, C₂-C₆alkenyl, C₃-C₆cycloalkyl and the like) are independently optionally substituted with one or more substituents independently selected from: halogen, hydroxyl, amino, oxo, mercapto, amido, cyano, azido, nitro, C₁-C₃alkyl, C₂-C₄alkenyl, C₂-C₄alkynyl, C₃-C₆cycloalkyl, C₁-C₄alkoxy. It should be noted that the radical positions on any molecular moiety used in the definitions may be anywhere on such a moiety as long as it is chemically permitted and stable.

The term “subject” represents any mammals including humans. In one embodiment of the invention, the subject is human.

The term “host” as used herein refers to a multicellular organism in which virus can replicate, including animals and preferably a human.

The term “independently” is used herein to indicate that the variable, which is independently applied, varies independently from application to application.

The invention also relates to methods of making the compounds of the invention.

The compounds may be prepared by any of the applicable methods and techniques of organic synthesis. Many such methods and techniques are well known in the art and some of the known methods techniques are elaborated in Compendium of Organic Synthetic Methods, in 12 volumes (John Wiley & Sons, New York); Advanced Organic Chemistry, 5 ed. M. Smith & J. March (John Wiley & Sons, New York, 2001); Comprehensive Organic Synthesis. Selectivity. Strategy & Efficiency in Modern Organic Chemistry, in 9 Volumes. Barry M. Trost, Editor-in-Chief (Pergamon Press, New York, 1993) and Chemistry of Nucleosides and Nucleotides, Townsend, L. B., Ed. (Plenum Press; New York, 1988).

A number of exemplary methods for the preparation of the compounds of the invention are provided below. These methods are intended to illustrate the nature of such preparations and are not intended to limit the scope of applicable methods. Alternative routes, which will be readily apparent to the ordinary skilled organic chemist, may alternatively be used to synthesize the compounds of the invention or their intermediates as illustrated by the general schemes and the preparative examples below.

In the course of the process described below for the preparation of compounds of Formula (I), functional groups in starting materials which are prone to participate in undesired side reactions, especially amino, amide, carboxy, hydroxy, phosphate, and mercapto groups, may be protected by suitable conventional protecting groups which are customarily used in the organic synthesis. Those protecting groups may already be present in the precursors and they are intended to protect the functional groups in question against undesired secondary reactions, such as acylation, etherification, esterification, alkylation, oxidation, reduction, solvolysis, etc. In certain cases the protecting groups can additionally cause the reactions to proceed selectively, for example regio selectively or stereoselectively. It is characteristic of protecting groups that they can be removed easily, i.e. without undesired secondary reactions taking place, for example by acid treatment, fluoride treatment, solvolysis, reduction, or by photolysis. The protection of functional groups by such protecting groups, the protecting groups themselves, and the reactions for their removal are described, for example, in standard works such as T. W. Greene and P. G. M. Wuts “Protective Groups in Organic Synthesis” John Wiley & Sons, Inc., New York 1999.

The compounds of the invention may be prepared through two general routes, as illustrated by the Scheme 1 and Scheme 2.

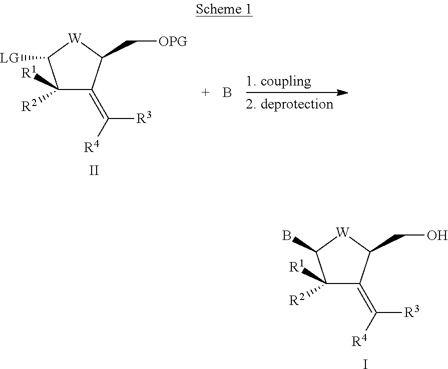

Scheme 1 describes a general method for the preparation of compounds according to Formula (I). The appropriate L-pentofuranosides, or L-4-thiopentofuranosides or cyclopentanes, wherein LG is a leaving group and R¹, R², R³, R⁴ and W are as defined for Formula (I) and are optionally properly-protected wherever necessary, are coupled with the optionally properly-protected nucleobases to obtain the compounds of Formula (I) after the deprotection. For example, the coupling reaction may be performed under Vorbrueggen coupling condition (H. Vorbrueggen, Acta Biochimica Polonica, 1996, 43, 26) where the per-silylated nucleobase is reacted with L-pentofuranosides, L-4-thiopentofuranosides under the catalysis such as TMS-triflate or other Lewis acids in an inert solvent or a mixture of inert solvents, such as acetonitrile, dichloromethane, chloroform, THF, and toluene. The common leaving groups at the 1-position of the L-pentofuranoside or L-4-thiopentofuranoside include alkoxy, acyl, halogen, in particular, methoxy, acetyl, chlorine or bromine (L. Wilson et al, Synthesis, 1995, 1465; J. Secrist III et al, J. Med. Chem. 1992, 35, 533; M. Dyson et al, J Med. Chem. 1991, 34, 2782; B. Huang et al, Nucleosides & Nucleotides 1993, 12, 139, M. Yokoyama, Synthesis, 2000, 1637). Alternatively, a sodium salt of purine may be used to couple with the compounds of formula (II) wherein the leaving group is a halide. (G. Revankar, Nucleosides Nucleotides 1989, 8, 709; C. Hildebrand et al, J. Org. Chem. 1992, 57, 1808). For the coupling between a nucleobase and a cyclopentane, the common leaving group include triflate, tosylate mesylate or halide. Alternatively, Mitsunobu reaction may be used for the coupling of a nucleobase and hydroxyl cyclopentanes (L. Agrofoglio et al, Tetrahedron 1994, 50, 10611, S. Schneller, Curr. Topics Med. Chem., 2002, 2, 1087).

Scheme 2 describes another method for the preparation of the compounds of Formula (I). The appropriately protected β-L-ribonucleosides, β-L-4′-thio-ribonucleosides or β-L-carbocyclic-ribonucleoside (III) can be transformed to the compounds of Formula (I) through several steps of reactions. The protecting groups on 2′, 3′ or 5′-hydroxyl may be different and can be selectively deprotected without affecting the protections at the other two sites.

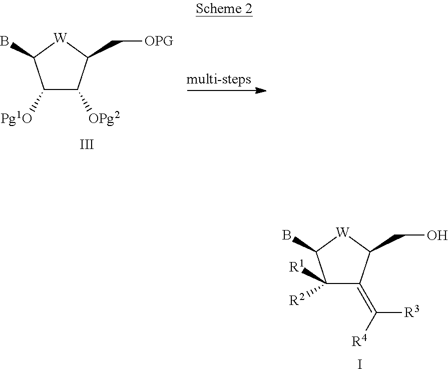

The transformation can be performed first on the 3′-hydroxyl group. After the selective protection, the 3′-hydroxyl group can be oxidized to a keto group using oxidation condition such as Dess-Martin reagent, CrO₃-pyridine-acetic anhydride, and the like. The keto group can be subsequently methylenated using applicable olefination conditions, for example Nysted reagent, Wittig reagents, Tebbe reagent, and so on (A. Matusda et al, Nucleosides & nucleotides 1992, 11, 197; M. Sharma et al, Tetrahedron Lett. 1990, 31, 5839; D. Lindegaard et al, Nucleosides, Nucleotides & Nucleic Acid, 2003, 22, 1159; P. Serafinowski et al, Tetrahedron Lett. 1996, 52, 7929; S. Auguste et al, J. Chem. Soc. Perkin Trans 1, 1995, 395; V. Samano et al, J. Org. Chem. 1991, 56, 7108). For preparation of 4′-thionucleosides, an approach via elimination reaction may be preferred. For example, a 3′-hydroxymethyl-4′ thio-nucleoside can be prepared and used as the key intermediate. The 3′-hydroxymethyl-4′-thionucleosides can be synthesized using the methods analogous to the literature methods (E. Ichikawa et al, Bioorg. Med. Che. Lett. 1999, 9, 1113; Braanalt et al, J. Org. Chem. 1994, 59, 4430; Moon et al, Bioorg. Med. Chem. Lett. 2002, 10, 1499; J. Sangvi et al, Synthesis, 1994, 1163; Sangvi et al, Tetrahedron Lett. 1994, 35, 4697; Mouldon, et al, Bioorg. Med. Chem. 1998, 6, 577; Faul et al, Tetrahedron, 1997, 53, 8085). The free hydroxyl group on the 3′-methyl can be sulphonylated, which is then subjected to a base treatment for elimination, leading to the 3′-methylidene compound. Alternatively, the free hydroxyl can be converted to iodide, which is subsequently subjected to elimination reaction. The bases used for the elimination may include sodium t-butoxide, potassium carbonate, cesium carbonate, triethylamine, DBU, DBN and the like. The resulting 3′-methylidene-β-L-nucleosides can be further modified at 2′-position to obtain the desired compounds of formula (I) using the methods known to the ordinary skilled nucleoside chemists (E. Ichikawa, Curr. Med. Chem., 2001, 8, 385; Chemistry of Nucleosides and Nucleotides, Townsend, L. B., Ed. (Plenum Press; New York, 1988)). Alternatively, the compounds of Formula (III) can be modified at 2′-position to obtain the intermediate with desired R¹ and R², which is subsequently followed by the introduction of 3′-methylidene group.

It is understood that some compounds of Formula (I) may be further modified to obtain desired compounds which can also be represented by Formula (I). The methods for such modifications depend on the structures of the desired products and the structure of the compound of formula (I) as the starting material. Such modification reaction may involve deprotection, substitution, addition, oxidation, reduction and other chemical transformations which are common in organic syntheses. For example, the compound of Formula (I) with R⁵ is H may be phosphorylated to a compound of Formula (I) with R⁵ is phosphate or triphosphate.

A number of exemplary methods for the preparation of compounds of the invention are provided herein, for example, in the Examples hereinbelow. These methods are intended to illustrate the nature of such preparations and are not intended to limit the scope of applicable methods. Certain compounds of the invention can be used as intermediates for the preparation of other compounds of the invention.

Scheme 3 describes a method for the preparation of some compounds of Formula (I) wherein W is oxygen and R³ and R⁴ are hydrogen. Tetraacetyl-L-ribofuranoside (IV) is condensed with per-silylated nucleobases such as uracil, thymine, cytosine, adenine, guanine or the properly protected nucleobases, under the catalysis of TMS-triflate, or other Lewis acids to obtain the β-L-ribonucleosides (V). The product was deacetylated under basic condition, such as sodium methoxide in methanol. The deprotected β-L-nucleosides can be selectively protected at 5′-hydroxyl and 2′-hydroxyl. The protecting groups on the 5′- and 2′-hydroxyl groups can be same or they can be different which can be selectively removed under proper deprotection condition. For example, both 2′-hydroxyl and 5′-hydroxyl can be protected by silyl protecting group like TBS group. Alternatively, the 5′-hydroxyl can be first protected by a trityl group such as trityl, 4-monomethoxytrityl or 4,4′-dimethoxytrityl. The 5′-protected nucleosides can be then selectively protected at 2′-hydroxyl, for example by t-butyldimethylsilyl group. The free 3′-hydroxyl group is converted to keto group by oxidation using the oxidation reagents, for example, Dess-Martin reagent or pyridine-chromiumoxide in acetic anhydride. The keto compounds (VII) are subsequently treated with olefination reagents such as Wittig reagent, Tebbe reagent or Nysted reagent, leading to 3′-deoxy-3′-methylidene-β-L-nucleosides (VIII). Compound of formula (VIII) can be directly deprotected to get compounds with R² being a hydroxyl (XI). Alternatively, after deprotection of the 2′-hydroxyl group, they can be deoxygenated via multi-steps and then deprotected to yield the compound of formula XII wherein both R¹ and R² are hydrogen. Alternatively, the 2′-hydroxyl group of Compounds IX can be inverted, leading to Compounds of formula X with R¹═OH and R²═H. Compounds of IX and X can be further derivatized to obtain compounds wherein R¹ and/or R² are independently H, F, CH₃, or OCH₃ using the methods known in the art.

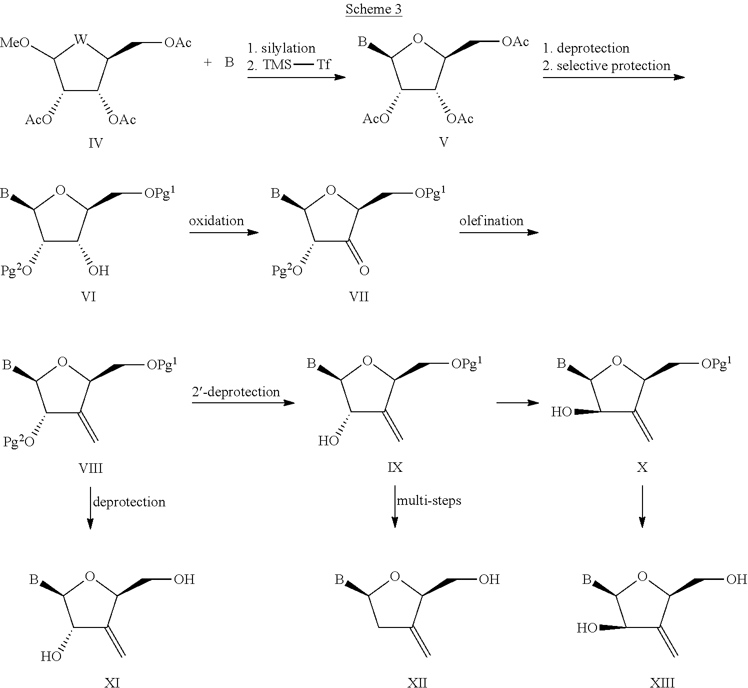

A number of exemplary procedures for the reaction are provided below. These methods are intended to illustrate the nature of such reactions and are not intended to limit the scope of methods. Alternative procedures, protocols or reaction conditions, which will be readily apparent to the ordinary skilled organic chemist, may alternatively be used.

### General Procedure A: Barton-McCombie Deoxygenation

To a solution of secondary alcohol (3.27 mmol) in dry 1,2-dichloroethane (10.9 mL) was added thio 10 (6.53 mmol) and the resultant yellow solution was heated to reflux for 1 h, cooled to room temperature and then poured into H₂O (7 mL). The phases were separated and the organic layer was washed with cold 1M HCl, then saturated aqueous NaHCO₃ solution and brine. The organic layer was then dried over MgSO₄ and concentrated under reduced pressure to give a pale yellow foam, which was dissolved in dry, degassed toluene (16.4 mL). Azacyclohexylcarbonitrile (0.327 mmol) and n-tributyltinhydride (6.53 mmol) was subsequently added and the resultant mixture was heated to reflux for 2 h and then concentrated under reduced pressure.

### General Procedure B: Dess-Martin Oxidation

To a suspension of Dess-Martin periodinane (2.10 mmol) in dry CH₂Cl₂ (20 mL) was added t-BuOH (2.31 mmol) and the resultant mixture was stirred at room temperature for 10 min before a solution of the secondary alcohol (1.75 mmol) in dry CH₂Cl₂ (6 mL) was added via cannula. The reaction mixture was stirred for 1.5 h at room temperature and then diluted with EtOAc (50 mL) and quenched with Na₂S₂O₃ (15 mL, 1M aq.), brine (10 mL) and NaHCO₃ (10 mL, sat. aq.). The biphasic mixture was stirred vigorously for 1 h and then the phases were separated. The aqueous layer was extracted once with EtOAc and the combined organic layers were dried (MgSO₄) and concentrated under reduced pressure to give pure ketone as a white foam, which was used without further purifications.

### General Procedure C: Nysted Olefination

To a suspension of Nysted's reagent (2.07 mmol, 20 w %) in dry THF (2.7 mL) was added the ketone (1.64 mmol) in dry CH₂Cl₂ (2.7 mL) drop wise via cannula at −78° C. TiCl₄ (1.67 mmol, 1.0M in CH₂Cl₂) was then added drop wise at −78° C. and the reaction mixture was stirred for 1.5 h. The temperature was then allowed to slowly reach room temperature over night. The mixture was then poured into saturated NaHCO₃ aqueous solution and stirred vigorously for 30 minutes and then filtered through celite. The phases were separated and the aqueous layer was extracted twice with CH₂Cl₂ and the combined organic layer were washed with brine, dried (MgSO₄) and concentrated under reduced pressure.

### General Procedure D: Acidic TBS-Deprotection

A solution of TBS-ether (0.0656 mmol) in AcOH:THF:H₂O (2 mL, 2:1:1) was stirred for 19 h at room temperature and then concentrated under reduced pressure.

General Procedure E: Converting Nucleosides with Uracil Base to Cytosine Base

To a solution of a nucleoside with uracil base (0.0969 mmol) in dry CH₂Cl₂:pyridine (1.0 mL, 5:1) was added triflic anhydride (0.218 mmol, 1M in CH₂Cl₂) at 0° C. The reaction mixture was then stirred at room temperature for 3 h and then NH₃ (5.5 mL, 7M in MeOH) was added. The resultant orange solution was stirred for 17 h and concentrated under reduced pressure.

### General Procedure F: TBS Deprotection Using Fluoride and Purification

To a solution of the TBS-ether (0.0468 mmol) in MeOH (2.3 mL) was added NH₄F (0.468 mL, 0.5M in MeOH). The resultant mixture was heated to reflux for 6 h and then concentrated under reduced pressure. The residue was dissolved in CH₂Cl₂:H₂O and the phases were separated. The aqueous layer was washed twice with CH₂Cl₂. Activated charcoal was added in small portions to the aqueous phase until it is no longer UV-active (spotted on TLC-plate). The charcoal suspension was loaded onto a flash column and eluted with H₂O (50 mL) followed by H₂O:MeOH (50 mL, 1:1). The correct fractions (spotted on TLC-plate) were collected and concentrated under reduced pressure to give the pure product.

It will be appreciated that the amount of a compound of Formula (I) of the present invention required for use in treatment will vary not only with the particular compound selected but also with the route of administration, the nature of the condition for which treatment is required and the age, weight and condition of the patient and will be ultimately at the discretion of the attendant physician. In general however a suitable dose may be in the range of from about 0.005 to about 30 mg/kg of body weight per day, preferably in the range of 0.05 to 10 mg/kg/day.

The desired dose is conveniently presented in a single dose or as divided dose administered at appropriate intervals, for example as two, three, four or more doses per day. Dependent on the need of the treatment and/or prevention, the desired dose may also be, for example, once every two days, once every three days, or even once a week.

The compound is conveniently administered in unit dosage form; for example containing 0.5 to 1500 mg conveniently 1 to 1000 mg most conveniently 5 to 700 mg of active ingredient per unit dosage form.

The compounds of the invention will normally be administrated via the oral, parenteral, intravenous, intramuscular, subcutaneous or other injectable ways, buccal, rectal, vaginal, transdermal and/or nasal route and/or via inhalation, in the form of pharmaceutical preparations comprising the active ingredient or a pharmaceutically acceptable salt or prodrug or solvate thereof, or a solvate of such a salt, in a pharmaceutically acceptable dosage form. Depending upon the disorder and patient to be treated and the route of administration, the compositions may be administered at varying doses.

While it is possible that, for use in therapy, a compound of Formula (I) of the present invention may be administered as the raw chemical, it is preferable according to one embodiment of the invention, to present the active ingredient as a pharmaceutical composition. The invention thus further provides a pharmaceutical composition comprising a compound of Formula (I) or a pharmaceutically acceptable salt or prodrug thereof together with one or more pharmaceutically acceptable carriers. The carrier(s) must be “acceptable” in the sense of being compatible with the other ingredients of the formulation and not deleterious to the recipient thereof. According to one embodiment of the present invention, pharmaceutical formulations include but are not limited to those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods according to this embodiment include the step of bringing into association the active compound with liquid carriers or finely divided solid carriers or both and then, if necessary, shaping the product into the desired composition.

Pharmaceutical compositions suitable for oral administration are conveniently presented as discrete units such as capsules, cachets or tablets each containing a predetermined amount of the active ingredient; as a powder or granules. In another embodiment, the formulation is presented as a solution, a suspension or as an emulsion. The active ingredient is alternatively presented as a bolus, electuary or paste.

Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds of Formula (I) may be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

The above described formulations are adapted to give sustained release of the active ingredient.

The following examples are provided to illustrate various embodiments of the present invention and shall not be considered as limiting in scope.

### Abbreviations

### DIPEA N;N-diisopropylethylamine;

DMAP 4-dimethylaminopyridine;
DMP Dess-Martin periodinane;
DBU 2,3,4,6,7,8,9,10-octahydropyrimidol[1,2-a]azepine;
EtOAc ethyl acetate;
Et₃N triethylamine;
THF tetrahydrofuran;

### DMF N,N-dimethylformamide;

DCM dichloromethane;
iPrOH isopropanol;
LCMS liquid chromatography mass spectroscopy;
MeCN acetonitrile;
RT room temperature;
TBS t-butyldimethylsilyl;
TBSCl t-butyldimethylsilyl chloride;
TBAF tetrabutylammonium fluoride;
TLC thin layer chromatography;
TFA trifluoroacetic acid;
p-TSA p-toluenesulfonic acid;

### NMP N-methylpyrrolidone;

R_{f} retention factor;
DAST (diethylamino)sulfur trifluoride;
MeOH methanol;
Hex hexane;
Hep heptane;
TMS trimethylsilyl;
EtOH ethanol;
AcOH acetic acid;
Et₂O diethylether;
Im imidazole;
n-Bu normal butyl;
i-Pr isopropyl;
Me methyl;
Bz benzoyl;
Ac acyl;
Ac₂O acetic anhydride;
Tf₂O triflic anhydride;
DHP 3,4-dihydro-2H-pyran;
THP tetrahydropyranyl;
DMTrCl 4,4′-dimethoxytrityl chloride;
DMTr 4,4′-dimethoxytrityl;
app apparent

### DMEM Dulbecco's Modified Eagle Medium

FBS Fetal bovine serum

If there is any inconsistency between the chemical name of the exemplified chemical compound and corresponding structure of said example, then the chemical structure should be used for determining the chemical compound of said example.

### EXAMPLE 1

### 1-(2,3-dideoxyl-3-methylidene-β-L-pentofuranosyl)uracil (Compound 8)

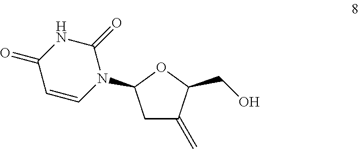

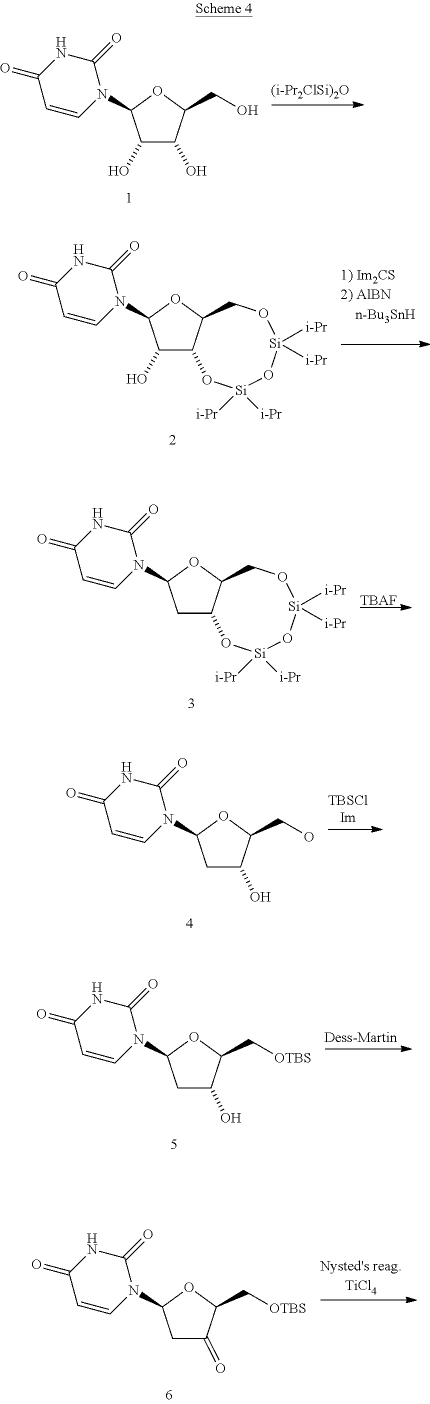

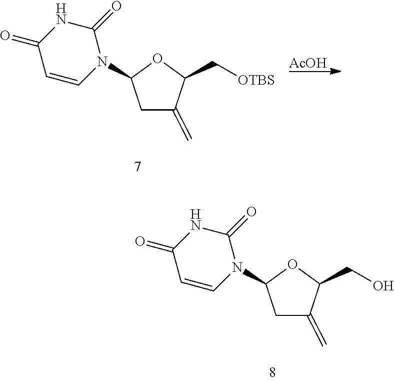

To a solution of β-L-uridine (Compound 1, 1.022 g, 4.18 mmol) in dry pyridine (8.4 mL) was added 1,3-dichloro-1,1,3,3-tetraisopropyldisiloxane (1.473 mL, 4.60 mmol) drop wise at 0° C. The resultant mixture was stirred at room temperature for 22 h and then concentrated under reduced pressure. The residue was dissolved in CH₂Cl₂ (50 mL) and washed three times with saturated NaHCO₃ aqueous solution. The combined aqueous layers were extracted with CH₂Cl₂. The combined organic layers were dried over MgSO₄ and concentrated under reduced pressure. Silica gel flash chromatography (CH₂Cl₂:EtOAc 4:1 to 1:1) of the residue gave Compound 2 (1.590 g) as a colorless foam.

Compound 2 (1.590 g, 3.27 mmol) was deoxygenized according to General Procedure A. Flash chromatography on silica gel (CH₂Cl₂:EtOAc 6:1 to 4:1) of the residue gave Compound 3 (1.127 g) as a white foam.

To a solution of Compound 3 (1.118 g, 2.38 mmol) in THF (7 mL) was added TBAF (4.78 mL, 4.78 mmol, 1M in THF) at 0° C. After 10 min, the temperature was allowed to reach room temperature and the mixture was stirred for 2.5 h and then concentrated under reduced pressure. Flash chromatography on silica gel (10 to 15% MeOH in CH₂Cl₂) of the residue gave Compound 4 (536 mg) as a white foam.

To a solution of Compound 4 (536 mg 2.35 mmol) in dry DMF (24 mL) was added TBSCl (372 mg 2.47 mmol) followed by imidazole (480 mg 7.05 mmol) at room temperature. After 3 h, the reaction mixture was poured into H₂O. The aqueous layer was extracted with EtOAc until TLC(R_{f}=0.67, CH₂Cl₂:MeOH 10:1) showed no product in the aqueous phase. The combined organic extracts were dried (MgSO₄) and concentrated under reduced pressure. Flash chromatography on silica gel (hexane:EtOAc 1:3) of the residue gave Compound 5 (598 mg) as a clear oil.

Compound 5 (598 mg 1.75 mmol) was oxidized according to General procedure B to give Compound 6 (561 mg), which was used without further purification.

Compound 6 (558 mg 1.64 mmol) was olefinated according to General procedure C. Flash chromatography (hexane:EtOAc 2:1) of the residue gave Compound 7 (161 mg) as a white solid.

Compound 7 (22.2 mg 0.0656 mmol) was deprotected according to General procedure D. Silica gel flash chromatography (5% MeOH in CH₂Cl₂) of the residue gave Compound 8 (9.5 mg) as a white solid. ¹H-NMR (400 MHz, CDCl₃) δ=7.72 (d, J=8.2 Hz, 1H), 6.23 (t, J=6.5 Hz, 1H), 5.74 (d, J=8.1 Hz, 1H), 5.24 (q, J=2.2 Hz, 1H), 5.08 (q, J=2.2 Hz, 1H), 4.59 (br s, 1H), 3.98 (dd, J=12.2, 2.7 Hz, 1H), 3.80 (dd, J=12.2, 3.9 Hz, 1H), 3.13 (m, 1H), 2.70 (ddq, J=16.6, 6.2, 2.3 Hz, 1H).

### EXAMPLE 2

### 1-(2,3-dideoxyl-3-methylidene-β-L-pentofuranosyl)cytosine (Compound 10)

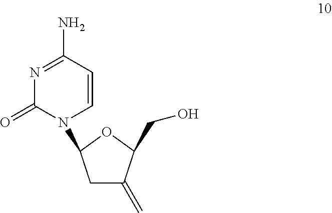

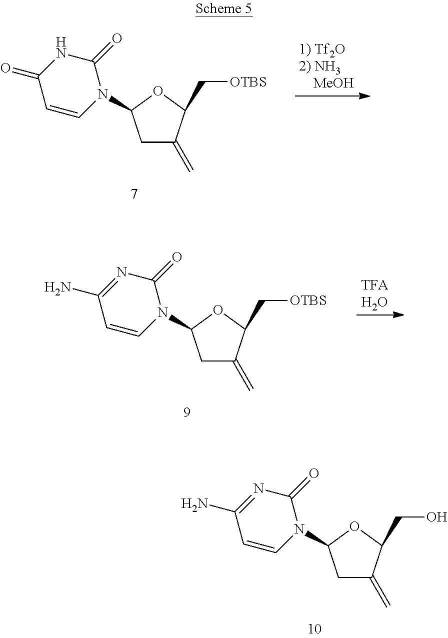

Compound 7 (32.8 mg 0.0969 mmol) was converted into the cytosine analogue (Compound 9) according to General procedure E. Flash chromatography on silica gel (5% MeOH in CH₂Cl₂) of the residue gave Compound 9 (24.8 mg) as a pale yellow oil.

To a solution of Compound 9 (13.2 mg 0.0391 mmol) in THF (2 mL) was added TFA:H₂O (1 mL, 1:1) at 0° C. After 1.25 h, the reaction mixture was concentrated under reduced pressure. The residue was dissolved in aqueous NaHCO₃ and was washed three times with CH₂Cl₂. Activated charcoal was added in small portions to the aqueous phase until it is no longer UV-active (spotted on TLC-plate). The charcoal suspension was loaded onto a flash column and eluted with H₂O (50 mL) followed by H₂O:MeOH (50 mL, 1:1). The product fractions were collected and concentrated under reduced pressure to give Compound 10 (5.0 mg) as a white solid. ¹H-NMR (500 MHz, MeOH-d₄) δ=8.03 (d, J=7.5 Hz, 1H), 6.16 (t, J=6.5 Hz, 1H), 5.92 (d, J=7.5 Hz, 1H), 5.19 (q, J=2.2 Hz, 1H), 5.09 (q, J=2.2 Hz, 1H), 4.56 (br s, 1H), 3.86 (dd, J=12.2, 3.1 Hz, 1H), 3.75 (dd, J=12.2, 4.4 Hz, 1H), 3.15 (dd, J=16.4, 6.2 Hz, 1H), 2.66 (m, 1H).

### EXAMPLE 3

### 1-(3-deoxy-3-methylidene-β-L-pentoribofuranosyl)uracil (Compound 15)

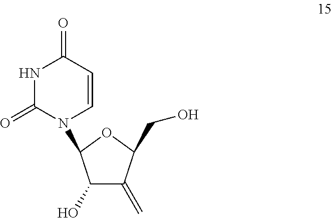

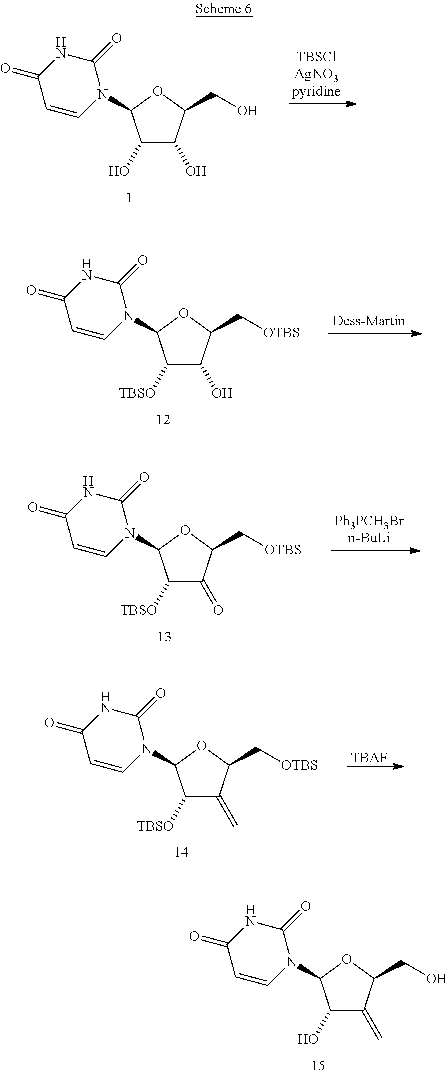

To a suspension of β-L-uridine (Compound 1,796 mg 3.26 mmol) in dry THF (100 mL) and dry pyridine (1.320 mL, 16.3 mmol) was added AgNO₃ (1.22 g, 7.17 mmol). After 5 min at room temperature, TBSCl (1.080 g, 7.17 mmol) was added and the resultant heterogeneous mixture was stirred at room temperature for 1 h. More AgNO₃ (554 mg 3.26 mmol) and TBSCl (490 mg 3.26 mmol) were added and the reaction mixture was stirred for 17 h at room temperature. The heterogeneous mixture was filtered through celite and diluted with CH₂Cl₂. The organic phase was washed with 1M HCl, sat. NaHCO₃, brine and was dried (MgSO₄) and concentrated under reduced pressure. Flash chromatography (Et₂O:Hex 1:1 to 2:1) of the residue gave Compound 12 (1.095 g) as a clear oil.

Compound 12 (438 mg 0.93 mmol) was oxidized according to General procedure B to give Compound 13, which was used without further purification.

To a suspension of methyltriphenylphosphonium bromide (995 mg 2.78 mmol) in dry THF (14 mL) was added n-BuLi (1.11 mL, 2.78 mmol, 2.5M in hexanes) at −78° C. After 1 h, the temperature was increased to 0° C. and the orange/red solution was stirred for 20 min before it was cooled to −78° C. A solution of Compound 13 (436 mg 0.93 mmol) in dry THF (9.5 mL) was added via cannula. After 30 min at −78° C., the temperature was increased to 0° C. for 30 min before the reaction mixture was stirred at room temperature for 15 h. The reaction mixture was then quenched with sat. aq. NH₄Cl and extracted twice with EtOAc. The combined organic layers were washed with brine, dried (MgSO₄) and concentrated under reduced pressure. Flash chromatography on silica gel (Et₂O:Hex 1:1) of the residue gave Compound 14 (410.5 mg) as a clear oil.

To a solution of Compound 14 (243 mg 0.518 mmol) in THF (5.2 mL) was added TBAF (1.56 mL, 1.56 mmol, 1M in THF) at room temperature. After 5 h, the reaction mixture was concentrated under reduced pressure. Flash chromatography on silica gel (4% MeOH in EtOAc) of the residue gave Compound 15 (122.5 mg) as a white solid.

### EXAMPLE 4

### 1-(3-deoxy-3-methylidene-β-L-pentoribofuranosyl)cytosine (Compound 17)

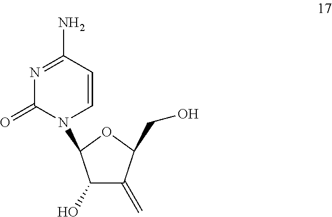

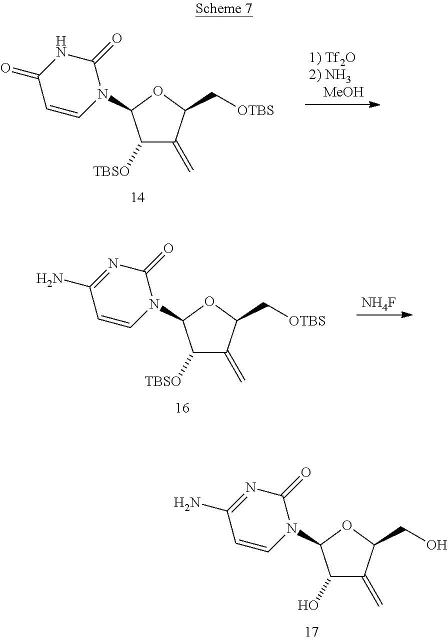

Compound 14 (56 mg 0.0119 mmol) was converted into the Compound 16 according to General procedure E. Flash chromatography on silica gel (5% MeOH in CH₂Cl₂) of the residue gave Compound 16 (51.6 mg) as a pale yellow oil.

Compound 16 (51.6 mg 0.110 mmol) was deprotected according to General procedure F. Activated charcoal purification (eluted with MeOH:H₂O 1:1) of the residue gave Compound 17 (15.5 mg) as a white solid. ¹H-NMR (500 MHz, MeOH-d₄) δ=8.00 (d, J=7.6 Hz, 1H), 5.98 (d, J=7.5 Hz, 1H), 5.85 (d, J=6.3 Hz, 1H), 5.36 (t, J=2.2 Hz, 1H), 5.24 (t, J=2.2 Hz, 1H), 4.69 (m, 1H), 4.62 (m, 1H), 3.84 (dd, J=12.1, 2.9 Hz, 1H), 3.72 (dd, J=12.1, 3.6 Hz, 1H).

### EXAMPLE 5

### 1-(3-deoxy-3-methylidene-β-L-arabinopentofuranosyl)uracil (Compound 21)

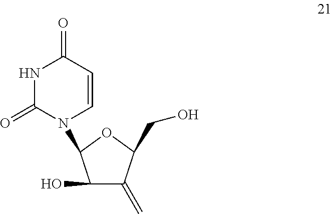

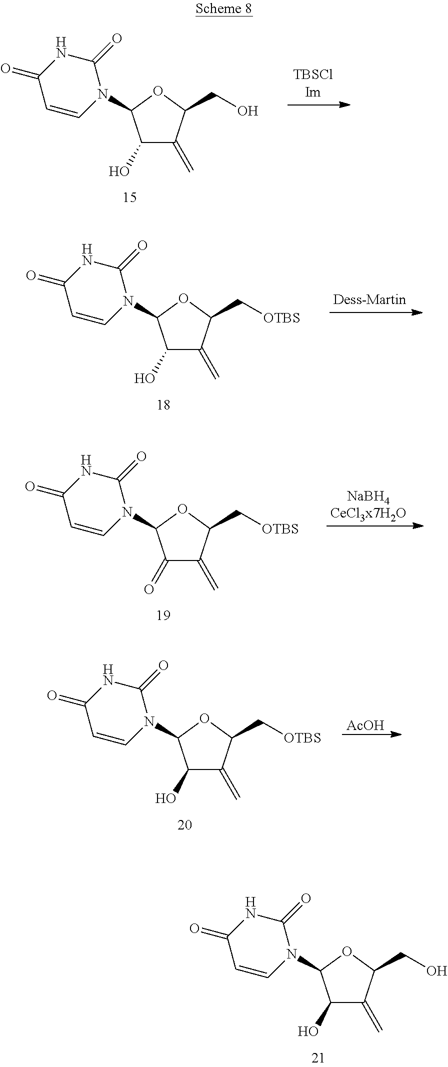

To a solution of Compound 15 (122.5 mg 0.510 mmol) in dry DMF (5.1 mL) was added TBSCl (92.2 mg 0.610 mmol) and imidazole (173.6 mg 2.55 mmol) at room temperature. After 5.5 h, the reaction mixture was diluted with Et₂O and H₂O. The phases were separated and the aqueous layer was extracted twice with Et₂O. The combined organic layers were washed three times with H₂O, dried (MgSO₄) and concentrated under reduced pressure. Flash chromatography (Hex:EtOAc 1:2) of the residue gave Compound 18 (110.5 mg) as a white solid.

Compound 18 (111 mg 0.313 mmol) was oxidized according to General procedure B to give Compound 19 (110 mg), which was used without further purification.

To a solution of 19 (110 mg 0.313 mmol) in MeOH (3.1 mL) was added CeCl₃×7H₂O (116.3 mg 0.313 mmol) and the solution was stirred at RT for 10 min. NaBH₄ (17.8 mg 0.470 mmol) was added in one portion and the resultant mixture was stirred at RT for 3 h and then quenched with sat. NH₄Cl and diluted with Et₂O. The layers were separated and then aqueous layer was extracted twice with Et₂O, dried (MgSO₄) and reduced under reduced pressure. Flash chromatography (Hex:EtOAc 1:2) of the residue gave 20 (42.5 mg) as a white solid.

To a solution of 20 (16.0 mg 0.0451 mmol) in THF (1 mL) was added TBAF (90 μL, 0.090 mmol, 1M in THF), stirred 2 h at RT and concentrated under reduced pressure. Flash chromatography (4% MeOH in EtOAc) of the residue gave 21 (10.2 mg) as a white solid. ¹H-NMR (500 MHz, MeOH-d₄) δ=7.84 (d, J=8.1 Hz, 1H), 6.05 (d, J=4.8 Hz, 1H), 5.62 (d, J=8.1 Hz, 1H), 5.47 (t, J=1.9 Hz, 1H), 5.30 (t, J=1.9 Hz, 1H), 4.68 (d, J=4.7 Hz, 1H), 4.57 (m, 1H), 3.85 (dd, J=12.1, 3.3 Hz, 1H), 3.79 (dd, J=12.1, 5.0 Hz, 1H).

### EXAMPLE 6

### 1-(2,3-dideoxy-3-methylidene-β-L-pentofuranosyl)thymine (Compound 26)

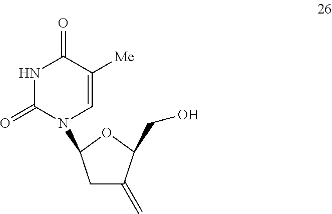

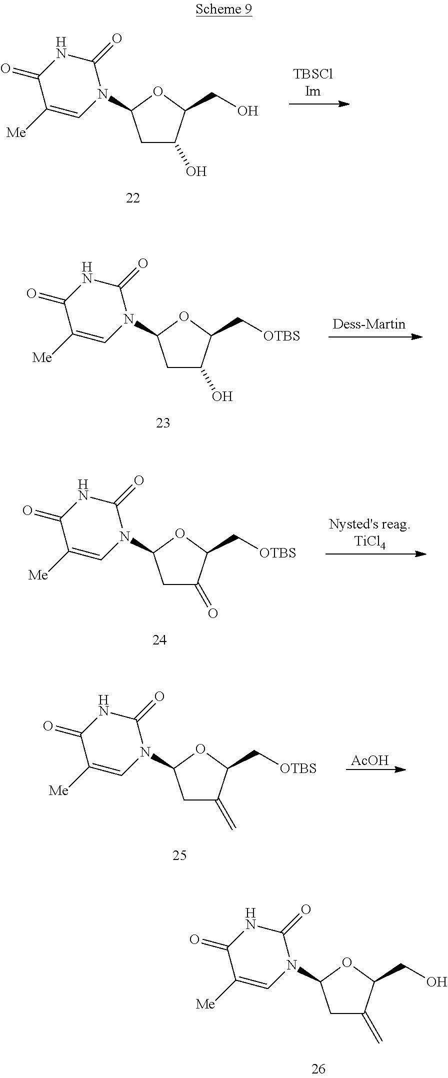

To a solution of β-L-thymidine (Compound 22,500 mg 2.06 mmol) in dry DMF (10 mL) was added TBSCl (342 mg 2.27 mmol) and imidazole (421 mg 6.18 mmol) at room temperature. After 20 h, the reaction mixture was diluted with Et₂O and H₂O. The phases were separated and the aqueous layer was extracted twice with Et₂O. The combined organic layers were washed three times with H₂O, dried (MgSO₄) and concentrated under reduced pressure. Flash chromatography (Hex:EtOAc 1:3) of the residue gave Compound 23 (602.6 mg) as a white solid.

Compound 23 (603 mg 1.69 mmol) was oxidized according to General procedure B to give Compound 24 (600 mg), which was used without further purification.

Compound 24 (600 mg 1.69 mmol) was olefinated according to General procedure C. Flash chromatography (hexane:EtOAc 2:1) of the residue gave Compound 25 (161 mg) as a white solid

Compound 25 (30.2 mg 0.0857 mmol) was deprotected according to General procedure D. Flash chromatography on silica gel (5% MeOH in CH₂Cl₂) of the residue gave Compound 26 (17.3 mg) as a white solid. ¹H-NMR (500 MHz, CDCl₃) δ=9.29 (br s, 1H), 7.46 (d, J=1.1 Hz, 1H), 6.22 (t, J=6.7 Hz, 1H), 5.22 (q, J=2.2 Hz, 1H), 5.07 (q, J=2.2 Hz, 1H), 4.57 (br s, 1H), 3.96 (dd, J=12.2, 2.7 Hz, 1H), 3.79 (dd, J=12.2, 4.2 Hz, 1H), 3.08 (dd, J=16.6, 6.8 Hz, 1H), 2.71 (ddq, J=16.5, 6.5, 2.2 Hz, 1H), 1.88 (d, J=1.1 Hz, 3H).

### EXAMPLE 7

### 1-(3-deoxy-3-methylidene-β-L-arabinopentofuranosyl)cytosine (Compound 28)

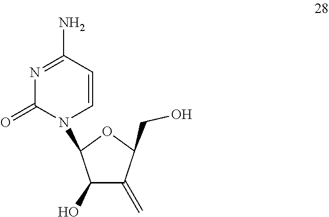

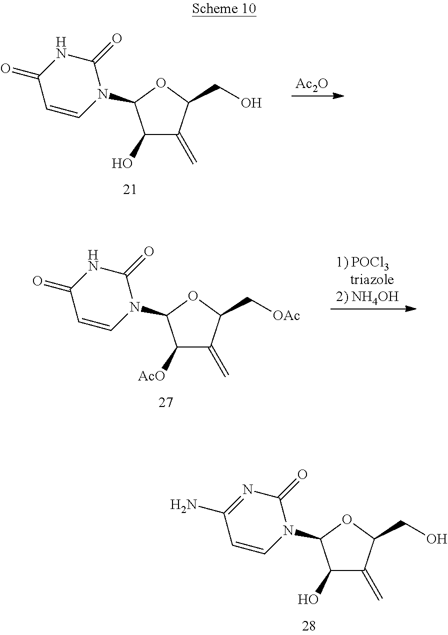

To a solution of Compound 21 (11.9 mg 0.0496 mmol) in dry pyridine (2 mL) was added acetic anhydride (1 mL) at RT. The resultant mixture was stirred for 2 h and then concentrated under reduced pressure. The residue was dissolved in CH₂Cl₂ and washed with saturated aqueous NaHCO₃ solution. The phases were separated and the aqueous phase was extracted twice with CH₂Cl₂. The combined organic phase were dried (MgSO₄) and concentrated under reduced pressure. Flash chromatography (Hex:EtOAc 1:2) of the residue gave Compound 27 (11.4 mg) as a colorless solid.

To a solution of Compound 27 (11.4 mg 0.0352 mmol), triazole (36.4 mg 0.527 mmol) and Et₃N (98 μL, 0.704 mmol) in dry acetonitrile (1 mL) was added POCl₃ (13.1 μL, 0.141 mmol) at 0° C. and the temperature was allowed to reach RT. The resultant mixture was stirred for 16 h and then diluted with EtOAc and quenched with saturated aqueous NaHCO₃ solution. The phases were separated and the aqueous phase was extracted twice with EtOAc. The combined organic extracts were dried (MgSO₄) and concentrated under reduced pressure. The residue was dissolved in dioxane (2 mL) and 25% NH₄OH (0.5 mL) was added and the reaction mixture was stirred for 24 h and then concentrated under reduced pressure. Flash chromatography (15% MeOH in CH₂Cl₂) of the residue gave Compound 28 (5.9 mg) as a colorless solid.

¹H NMR (500 MHz, MeOH-d₄) δ=7.85 (d, J=7.5 Hz, 1H), 6.04 (d, J=4.3 Hz, 1H), 5.84 (d, J=7.5 Hz, 1H), 5.47 (dd, J=1.9, 1.2 Hz, 1H), 5.30 (app. t, J=1.4 Hz, 1H), 4.63 (d, J=4.3 Hz, 1H), 4.60-4.55 (m, 1H), 3.84 (dd, J=11.9, 3.3 Hz, 1H), 3.78 (dd, J=12.0, 5.2 Hz, 1H).

### EXAMPLE 8

### 1-[2-deoxy-2-(S)-fluoro-3-deoxy-3-methylidene-β-L-pentofuranosyl]uracil (Compound 35)

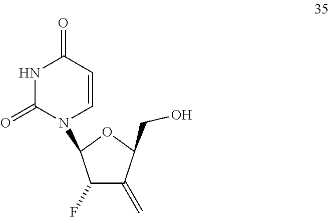

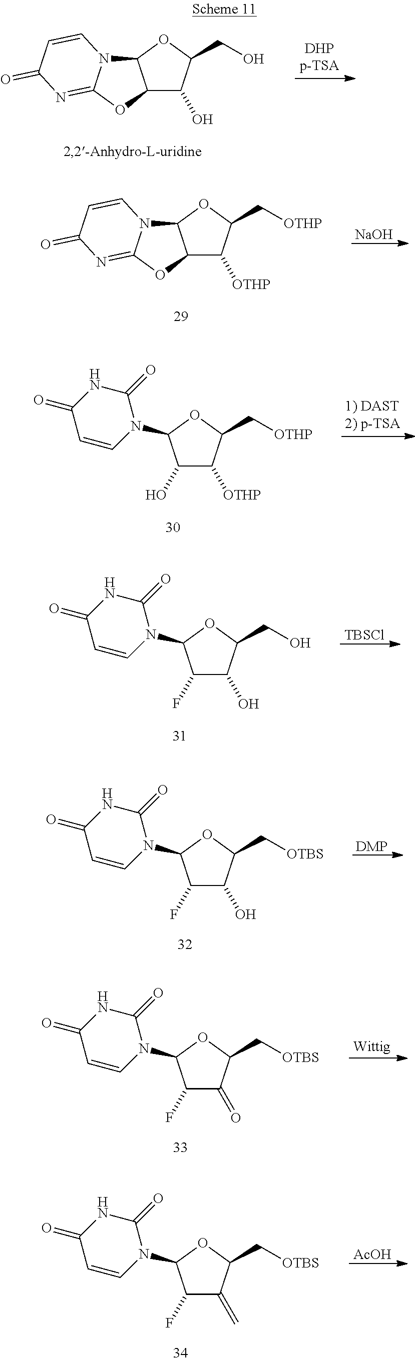

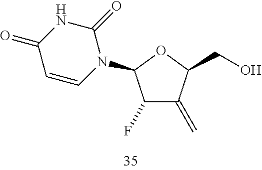

To a solution of 2,2′-anhydro-L-uridine (300 mg 1.33 mmol) and 3,4-dihydropyran (3.2 mL) in dry DMF (5.3 mL) was added p-TSA (250 mg) at 0° C. The reaction mixture was stirred at RT for 3 h and then quenched with Et₃N (550 μL). The mixture was concentrated under reduced pressure, dissolved in EtOAc and washed with saturated aqueous NaHCO₃ solution, brine, dried (MgSO₄) and concentrated. Trituration of the crude product with hexane gave Compound 29 (418 mg) as a colorless solid.

To a solution of Compound 29 (418 mg 1.06 mmol) in MeOH (10 mL) was added NaOH (1.8 mL, 1M in MeOH) and the reaction mixture was stirred at RT for 2.5 h. The reaction was quenched with AcOH (100 μL) and then concentrated under reduced pressure. Flash chromatography (EtOAc) of the residue gave Compound 30 (435 mg) as a colorless oil.

To a solution of Compound 30 (231 mg 0.56 mmol) in dry CH₂Cl₂:pyridine (5.6 mL, 6:1) was added DAST (230 μL, 1.74 mmol) at 0° C. under N₂. The reaction mixture was heated to reflux for 5 h and then cooled down to room temperature and quenched with saturated aqueous NaHCO₃ solution. The mixture was extracted twice with CH₂Cl₂, washed with saturated aqueous NaHCO₃ solution, dried (MgSO₄) and concentrated under reduced pressure. The residue was dissolved in MeOH (5.6 mL) and p-TSA (107 mg 0.56 mmol) was added. The reaction mixture was stirred at RT for 5 h and then concentrated under reduced pressure. Flash chromatography (CH₂Cl₂:MeOH 10:1) of the residue gave Compound 31 (91.8 mg) as a colorless oil.

To a solution of Compound 31 (78.4 mg 0.318 mmol) in dry DMF (3.2 mL) was added TBSCl (50.4 mg 0.334 mmol) and imidazole (64.9 mg 0.954 mmol) at 0° C. The reaction mixture was stirred at this temperature for 1.5 h and was then quenched with H₂O. The mixture was extracted three times with Et₂O and the combined organic extracts was washed three times with H₂O, dried (MgSO₄) and concentrated under reduced pressure. Flash chromatography (hexane:EtOAc 1:1) of the residue gave Compound 32 (86.7 mg) as a solid.

Compound 32 (64.7 mg 0.179 mmol) was oxidized according to General procedure B to give Compound 33 (64.3 mg), which was used without further purification.

To a suspension of methyltriphenylphosphonium bromide (192 mg 0.538 mmol) in dry THF (2.7 mL) was added n-BuLi (0.215 mL, 0.538 mmol, 2.5M in hexanes) at −78° C. After 0.5 h, the temperature was increased to 0° C. and the orange/red solution was stirred for 20 min. then it was re-cooled to −78° C. Thereafter, a solution of Compound 33 (64.3 mg 0.179 mmol) in dry THF (1.8 mL) was added via cannula. After 2 h at −78° C., the reaction mixture was quenched with saturated aqueous NH₄Cl solution and extracted twice with Et₂O. The combined organic layers were washed with brine, dried (MgSO₄) and concentrated under reduced pressure. Flash chromatography on silica gel (Et₂O:Hex 1:1) of the residue gave Compound 34 (36.1 mg) as a colorless solid.

Compound 34 (9.5 mg 0.0267 mmol) was deprotected according to General procedure D. Flash chromatography (5% MeOH in CH₂Cl₂) of the residue gave Compound 35 (6.5 mg) as a colorless oil.

¹H NMR (500 MHz, MeOH-d₄) δ=7.89 (d, J=8.1 Hz, 1H), 6.07 (dd, J=16.3, 3.3 Hz, 1H), 5.69 (d, J=8.1 Hz, 1H), 5.63-5.61 (m, 1H), 5.50 (ddd, J=54.4, 3.2, 1.5 Hz, 1H), 5.49-5.46 (m, 1H), 4.79 (d, J=1.7 Hz, 1H), 3.90 (dd, J=12.3, 2.9 Hz, 1H), 3.79 (dd, J=12.3, 3.8 Hz, 1H).

### EXAMPLE 9

### 1-[2-deoxy-2-(S)-fluoro-3-deoxy-3-methylidene-β-L-pentofuranosyl]cytosine (Compound 37)

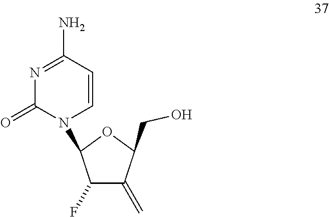

Compound 34 (25.6 mg 0.0718 mmol) was deprotected according to General procedure D. The residue was azeotropically dried with pyridine (2×2 mL) and then dissolved in dry pyridine (2 mL). To the resultant solution was added acetic anhydride (0.5 mL). The reaction mixture was stirred at RT for 4 h and then concentrated under reduced pressure. Flash chromatography (hexane:EtOAc 1:2) of the residue gave Compound 36 (13.7 mg) as a colorless oil.

To a solution of Compound 36 (13.7 mg 0.0482 mmol), 1,2,4-triazole (50.0 mg 0.723 mmol) and Et₃N (135 μL, 0.964 mmol) in dry MeCN (1 mL) was added POCl₃ (18.0 μL, 0.193 mmol) at 0° C. The resultant mixture was stirred at RT for 4 h, quenched with saturated aqueous NaHCO₃ solution and extracted three times with CH₂Cl₂. The combined organic extracts were dried (MgSO₄) and concentrated under reduced pressure. The residue was dissolved in NH₃ (3 mL, 7 N in MeOH) and stirred at RT for 20 h followed by 50° C. for 2 h and then concentrated under reduced pressure. The residue was purified to give Compound 37 (4.2 mg).

¹H NMR (500 MHz, MeOH-d₄) δ=7.90 (d, J=7.5 Hz, 1H), 6.04 (dd, J=16.5, 3.0 Hz, 1H), 5.88 (d, J=7.5 Hz, 1H), 5.63-5.59 (m, 1H), 5.47-5.45 (m, 1H), 5.44 (ddd, J=54.4, 2.8, 1.3 Hz, 1H), 4.82-4.77 (m, 1H), 3.92 (dd, J=12.3, 3.0 Hz, 1H), 3.81 (dd, J=12.4, 3.9 Hz, 1H).

### EXAMPLE 10

### 1-[2-deoxy-2-(R)-fluoro-3-deoxy-3-methylidene-β-L-pentofuranosyl]uracil (Compound 45)

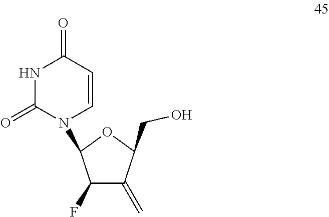

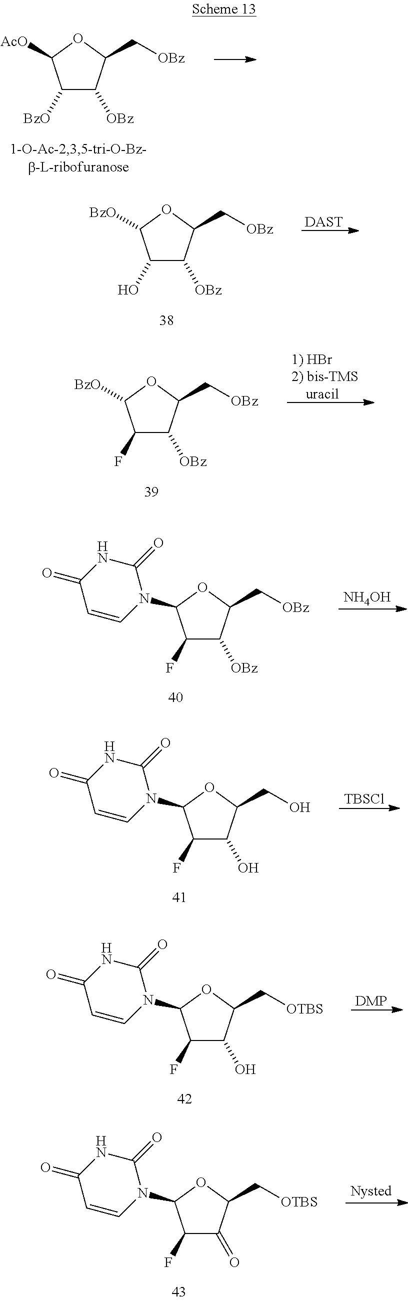

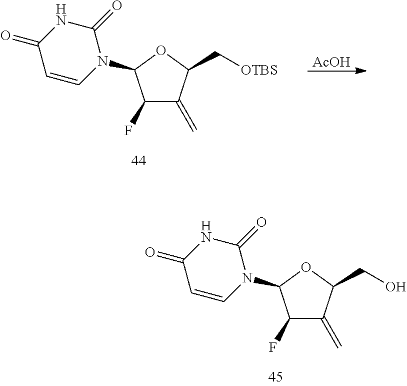

To a solution of 1-O-acetyl-2,3,5-tri-O-benzoyl-β-L-ribofuranose (3.00 g, 5.95 mmol) and acetyl bromide (0.68 mL, 9.22 mmol) in dry CH₂Cl₂ (15 mL) was added dry MeOH (0.36 mL) at 0° C. The reaction mixture was stirred at this temperature for 2 h and then H₂O (6 mL) was added and the mixture was vigorously stirred at RT for 2 h. The phases were separated and the aqueous phase was extracted with CH₂Cl₂, dried (MgSO₄) and concentrated under reduced pressure to approx. 10-15 mL. The residue was cooled to 0° C. and heptane (20 mL) was added under stirring. The solution was concentrated on a rotavapor (no water bath) until precipitation occurred. The mixture was then stirred under atmospheric pressure at 0° C. for 30 min. The precipitate was filtered off and washed with 3×3 mL (hep:CH₂Cl₂ 2:1) and dried in vacuo to give Compound 38 (1.275 g) as a colorless solid.

To a solution of Compound 38 (1.28 g, 2.76 mmol) in dry CH₂Cl₂ (18.4 mL) was added DAST (1.09 mL, 8.27 mmol) at RT under N₂. The reaction mixture was heated to reflux for 18 h and then DAST (0.50 mL, 4.14 mmol) was added and the mixture was stirred at reflux for 6 h and then quenched with saturated aqueous NaHCO₃ solution (10 mL). The phases were separated and the organic phase was washed with H₂O, aqueous NaHCO₃ solution, dried (MgSO₄) and concentrated under reduced pressure. Silica gel flash chromatography (CH₂Cl₂) of the residue gave Compound 39 (927 mg) as a colorless solid.

To a solution of Compound 39 (927 mg 2.00 mmol) in dry CH₂Cl₂ (5 mL) was added HBr (1.16 mL, 4.28 mmol, 33% in AcOH) under N₂. And the resultant mixture was stirred under N₂ for 17 h. The reaction mixture was washed with twice H₂O and NaHCO₃, dried (MgSO₄) and concentrated under reduced pressure to give the crude bromide (838 mg) as a pale yellow oil. In a separate flask, uracil (269 mg 2.40 mmol) and (NH₄)₂SO₄ (16 mg) in hexamethyldisilazane (5.0 mL) was heated to reflux under N₂ for 22 h. The reaction mixture was concentrated under reduced pressure and dried under high vacuum to give the crude bis-TMS-uracil as a colorless oil. The crude bromide in dry CHCl₃ (10 mL) was added to crude bis-TMS-uracil via cannula under N₂. The resultant mixture was heated to reflux under N₂ for 18 h and then quenched with H₂O and stirred at RT for 30 min. The phases were separated and the aqueous phase was extracted twice with CH₂Cl₂. The combined organic extracts were dried (MgSO₄) and concentrated under reduced pressure. Recrystallization from EtOH gave Compound 40 (598 mg) as a colorless solid.

To a solution of Compound 40 (527 mg 1.16 mmol) in MeOH (8.1 mL) was added 25% NH₄OH and the resultant mixture was stirred at RT for 41 h and then concentrated under reduced pressure. Silica gel flash chromatography (CH₂Cl₂:MeOH 10:1) of the residue gave Compound 41 (275 mg) as a colorless solid.

To a solution of Compound 41 (280 mg 1.14 mmol) in dry DMF (11.4 mL) was added TBSCl (180.2 mg 1.20 mmol) and imidazole (232 mg 3.42 mmol) at 0° C. The reaction mixture was slowly allowed to reach RT and stirred for 16 h and was then quenched with H₂O. The mixture was extracted three times with Et₂O and the combined organic extracts were washed three times with H₂O, dried (MgSO₄) and concentrated under reduced pressure. Flash chromatography (hexane:EtOAc 1:2) of the residue gave Compound 42 (365) as a colorless solid.

Compound 42 (281 mg 0.778 mmol) was oxidized according to General procedure B. The reaction mixture was diluted with EtOAc and then quenched with Na₂S₂O₃ (1.65 g) in pH 7.4 buffer (11 mL, 0.1M) and stirred vigorously until the suspension became clear. The phases were separated and the organic phase was washed (10 s) with NaHCO₃ (5% aqueous solution), dried (MgSO₄) and concentrated under reduced pressure to give Compound 43 (279 mg 100%) as a colorless solid, which was used without further purification.

Compound 43 (279 mg 0.778 mmol) was olefinated according to General procedure C. Silica gel flash chromatography (hexane:EtOAc 2:1) of the residue gave Compound 44 (112 mg) as a colorless solid.

Compound 44 (23.5 mg 0.0656 mmol) was deprotected according to General procedure D. Silica gel flash chromatography (5% MeOH in CH₂Cl₂) of the residue gave Compound 45 (13.1 mg) as a colorless solid.

¹H NMR (500 MHz, MeOH-d₄) δ=7.88 (dd, J=8.1, 2.0 Hz, 1H), 6.11 (dd, J=17.5, 3.4 Hz, 1H), 5.76 (dd, J=6.5, 2.2 Hz, 1H), 5.69 (d, J=8.1 Hz, 1H), 5.56 (d, J=5.6 Hz, 1H), 5.38 (dd, J=55.7, 3.3 Hz, 1H), 4.65-4.60 (m, 1H), 3.84-3.75 (m, 2H).

### EXAMPLE 11

### 1-[2-deoxy-2-(R)-fluoro-3-deoxy-3-methylidene-β-L-pentofuranosyl]cytosine (Compound 47)

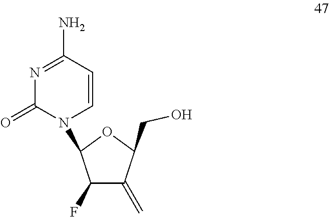

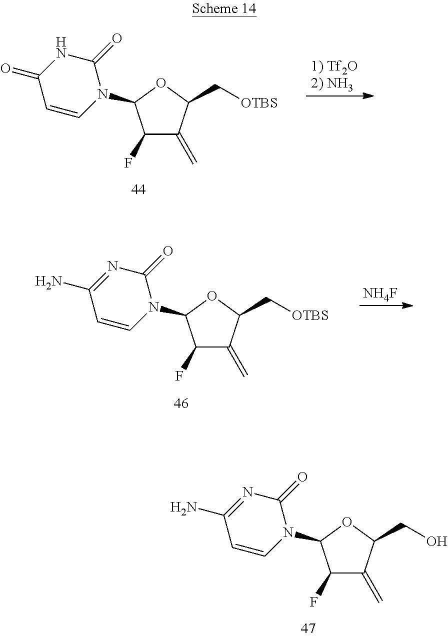

Compound 44 (86.7 mg 0.243 mmol) was converted into the cytidine analogue (Compound 46) according to General procedure E. Silica gel flash chromatography (5% MeOH in CH₂Cl₂) of the residue gave Compound 46 (63.9 mg) as a pale yellow oil.

Compound 46 (54 mg 0.152 mmol) was deprotected according to General procedure F. The residue was purified to give Compound 47 (34.7 mg).

¹H NMR (500 MHz, MeOH-d₄) δ=7.88 (d, J=7.5 Hz, 1H), 6.07 (dd, J=18.1, 2.9 Hz, 1H), 5.89 (d, J=7.6 Hz, 1H), 5.75 (d, J=6.6 Hz, 1H), 5.55 (d, J=5.4 Hz, 1H), 5.37 (dd, J=55.6, 2.5 Hz, 1H), 4.63 (s, 1H), 3.78 (d, J=5.0 Hz, 2H).

### EXAMPLE 12

### 1-[(2S,3S,5R)-5-(Hydroxymethyl)-3-methyl-4-methylenetetrahydrofuran-2-yl]pyrimidine-2,4(1H,3H)-dione (Compound 55)

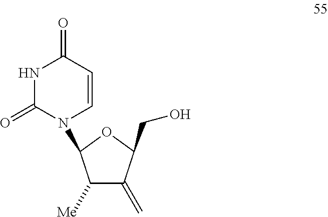

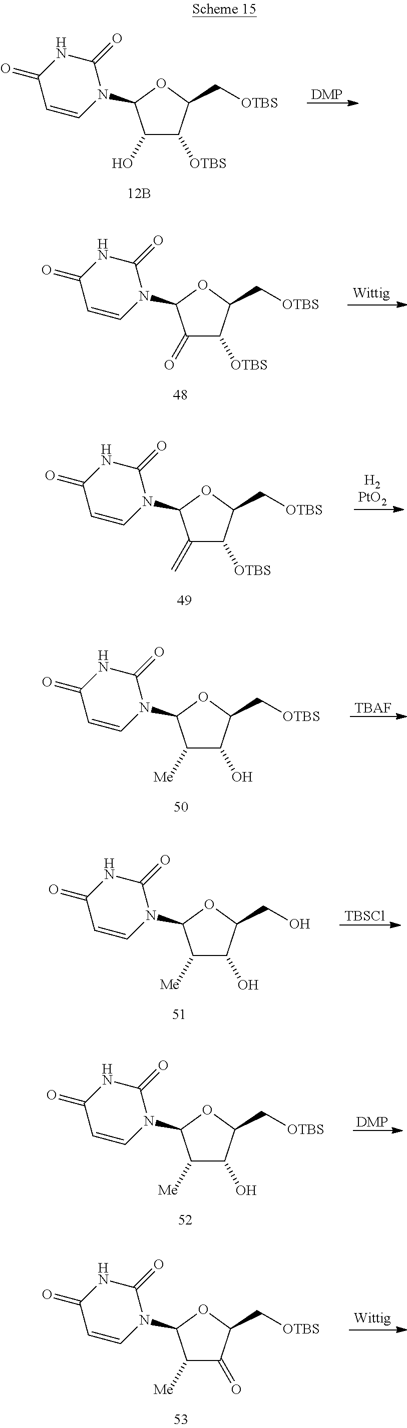

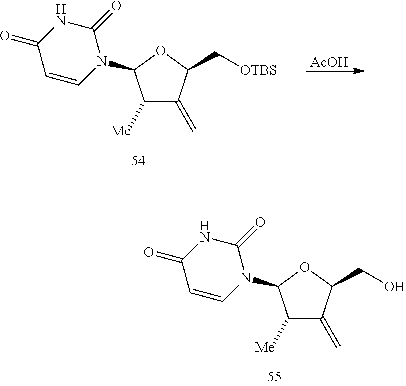

1-{(2S, 3S, 4R, 5S)-4-(tert-butyldimethylsilyloxy)-5-[(tert-butyldimethylsilyloxy)-methyl]-3-hydroxytetrahydrofuran-2-yl}pyrimidine-2,4(1H,3H)-dione (Compound 12B) (274 mg 0.580 mmol) was oxidized according to General procedure B to give Compound 48 (273 mg) as a colorless solid, which was used without further purification.

To a suspension of methyltriphenylphosphonium bromide (622 mg 1.74 mmol) in dry THF (8,7mL) was added n-BuLi (1.09 mL, 0.54 mmol, 1.6 M in hexanes) at −78° C. After 0.5 h, the temperature was increased to 0° C. and the orange/red solution was stirred for 20 min before it was re-cooled to −78° C. Thereafter, a solution of Compound 48 (64.3 mg 0.179 mmol) in dry THF (6 mL) was added via cannula. After 1 h at −78° C. the temperature was allowed to reach room temperature and the reaction mixture was stirred at this temperature for 20 h. The reaction mixture was then quenched with saturated aqueous NH₄Cl solution and extracted twice with Et₂O. The combined organic extracts were washed with brine, dried (MgSO₄) and concentrated under reduced pressure. Silica gel flash chromatography (Et₂O:Hexane 1:1) of the residue gave Compound 49 (74.6 mg) as a colorless solid.

A solution of Compound 49 (300 mg 0.640 mmol) and PtO₂ (14.5 mg 0.0640 mmol) in absolute EtOH (12.8 mL) was stirred under a H₂-atmosphere for 1 h at RT. The reaction mixture was filtered through glass wool and concentrated under reduced pressure to give Compound 50 (301 mg) that was used without further purification. To a solution of Compound 50 (301 mg 0.639 mmol) in THF (4.5 mL) was added TBAF (1.9 mL, 1.918 mmol, 1M in THF) at 0° C. The reaction mixture was stirred at RT for 2 h and then concentrated under reduced pressure. Silica gel flash chromatography (5% MeOH in EtOAc) of the residue gave Compound 51 (142.5 mg) as a colorless solid.

To a solution of Compound 51 (54.5 mg 0.225 mmol) in dry DMF (2.3 mL) was added TBSCl (35.6 mg 0.236 mmol) and imidazole (46.0 mg 0.675 mmol) at 0° C. The reaction mixture was slowly allowed to reach RT and stirred for 18 h and was then quenched with H₂O. The mixture was extracted three times with Et₂O and the combined organic extracts were washed three times with H₂O, dried (MgSO₄) and concentrated under reduced pressure. Silica gel flash chromatography (hexane:EtOAc 1:2) of the residue gave Compound 52 (67.6 mg) as a colorless oil.

Compound 52 (124 mg 0.348 mmol) was oxidized according to General procedure B. The reaction mixture was diluted with EtOAc and then quenched with Na₂S₂O₃ (0.75 g) in pH 7.4 buffer (5.1 mL, 0.1M) and stirred vigorously until the suspension became clear. The phases were separated and the organic phase was washed with NaHCO₃ (5% aqueous solution), dried (MgSO₄) and concentrated under reduced pressure to give Compound 53 (123 mg) as a colorless solid, which was used without further purification.

Compound 53 (123 mg 0.348 mmol) was olefinated according to General Procedure C. Silica gel flash chromatography (hexane:EtOAc 2:1) of the residue gave Compound 54 (38.5 mg) as a colorless solid.

Compound 54 (6.0 mg 0.0170 mmol) was deprotected according to General Procedure D. Silica gel flash chromatography (5% MeOH in CH₂Cl₂) of the residue gave Compound 55 (3.8 mg) as a colorless solid.

¹H NMR (500 MHz, MeOH-d₄) δ=7.98 (d, J=8.2 Hz, 1H), 5.75 (d, J=6.1 Hz, 1H), 5.74 (d, J=6.0 Hz, 1H), 5.13 (dd, J=2.8, 1.9 Hz, 1H), 5.10 (app. t, J=2.4 Hz, 1H), 4.58-4.54 (m, 1H), 3.83 (dd, J=12.0, 2.9 Hz, 1H), 3.73 (dd, J=12.0, 3.9 Hz, 1H), 2.85-2.76 (m, 1H), 1.18 (d, J=6.7 Hz, 3H).

### EXAMPLE 13

### 4-Amino-1-[(2S,3S,5R)-5-(hydroxymethyl)-3-methyl-4-methylenetetrahydrofuran-2-yl]pyrimidin-2(1H)-one (Compound 57)

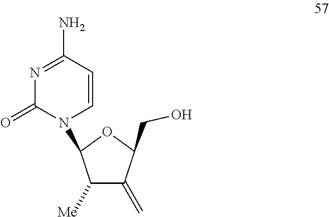

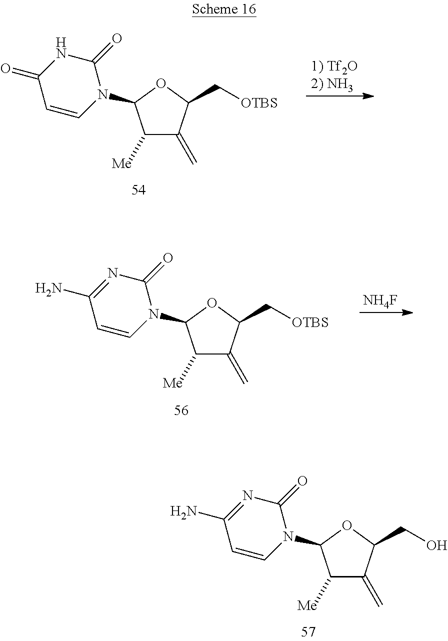

Compound 54 (15.0 mg 0.0425 mmol) was converted into the cytidine analogue (Compound 56) according to General Procedure E. Silica gel flash chromatography (5% MeOH in CH₂Cl₂) of the residue gave Compound 56 (11.0 mg) as a pale yellow oil.

Compound 56 (11.0 mg 0.0313 mmol) was deprotected according to General procedure F. The residue was purified to give Compound 57 (5.6 mg).

¹H NMR (500 MHz, MeOH-d₄) δ=7.97 (d, J=7.5 Hz, 1H), 5.94 (d, J=7.5 Hz, 1H), 5.82 (d, J=8.1 Hz, 1H), 5.11 (dd, J=2.8, 1.9 Hz, 1H), 5.09 (app. t, J=2.4 Hz, 1H), 4.56 (dd, J=3.3, 1.4 Hz, 1H), 3.83 (dd, J=12.0, 3.0 Hz, 1H), 3.73 (dd, J=12.0, 4.0 Hz, 1H), 2.82-2.69 (m, 1H), 1.18 (d, J=6.7 Hz, 3H).

### EXAMPLE 14

### 1-(2,3-dideoxyl-3-methylidene-β-L-pentofuranosyl)-5-fluorouracil (Compound 64)

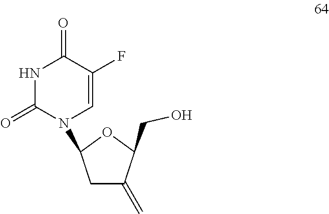

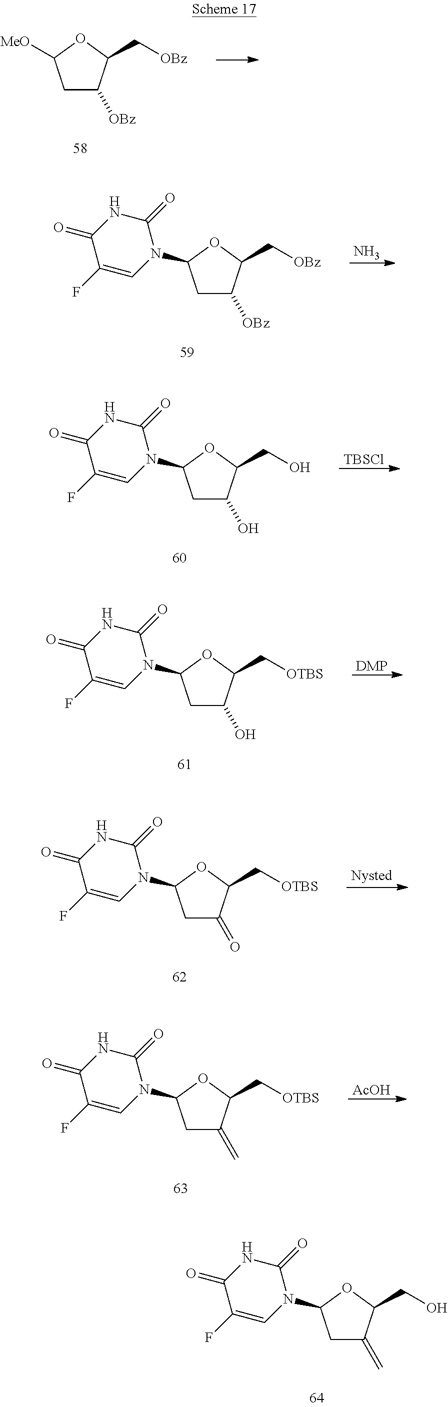

A solution of 5-fluorouracil (920 mg 7.07 mmol) and N,O -bis(trimethysilyl)acetamide (3.5 mL, 14.1 mmol) in dry MeCN (36 mL) was heated to reflux for 40 min. The reaction mixture was then cooled to 0° C. and Compound 58 (1.14 g, 3.20 mmol) and SnCl₄ (32.0 mL, 32.0 mmol, 1M in CH₂Cl₂) were added and the resultant mixture was stirred at RT under N₂ over night. The solution was diluted with EtOAc and poured into ice-cold saturated aqueous NaHCO₃ solution. The phases were separated and the aqueous phase was extracted twice with EtOAc. The combined organic extracts were washed with brine and filtered through a plug of silica gel (EtOAc) and concentrated under reduced pressure. Silica gel flash chromatography (CH₂Cl₂:EtOAc 1:0-12:1-8:1) of the residue gave Compound 59 (427 mg) as a colorless oil.

A solution of Compound 59 (384 mg 0.845 mmol) in NH₃ (10 mL, 7 N in MeOH) was stirred at RT for 24 h and then concentrated under reduced pressure. Flash chromatography (15% MeOH in CH₂Cl₂) of the residue gave Compound 60 (198 mg) as a colorless foam.

To a solution of Compound 60 (275 mg 1.12 mmol) in dry DMF (11 mL) was added TBSCl (177 mg 1.17 mmol) and imidazole (229 mg 3.36 mmol) at 0° C. The reaction mixture was slowly allowed to reach RT and stirred for 17 h and was then quenched with H₂O. The mixture was extracted three times with Et₂O and the combined organic extracts were washed three times with H₂O, dried (MgSO₄) and concentrated under reduced pressure. Silica gel flash chromatography (hexane:EtOAc 1:2) of the residue gave Compound 61 (260 mg) as a colorless solid.

Compound 61 (259 mg 0.719 mmol) was oxidized according to General procedure B to give Compound 62 (258 mg) as a colorless solid, which was used without further purification.

Compound 62 (258.0 mg 0.719 mmol) was olefinated according to General procedure C. Silica gel flash chromatography (hexane:EtOAc 2:1) of the residue gave Compound 63 (46.0 mg) as a colorless solid.

Compound 63 (18.3 mg 0.0513 mmol) was deprotected according to General procedure D. Flash chromatography (5% MeOH in CH₂Cl₂) of the residue gave Compound 64 (9.8 mg) as a colorless oil.

¹H NMR (500 MHz, MeOH-d₄) δ=8.21 (d, J=6.8 Hz, 1H), 6.17 (td, J=6.5, 1.8 Hz, 1H), 5.22 (q, J=2.2 Hz, 1H), 5.10 (q, J=2.2 Hz, 1H), 4.53 (s, 1H), 3.88 (dd, J=12.2, 2.8 Hz, 1H), 3.77 (dd, J=12.2, 3.7 Hz, 1H), 3.15-3.06 (m, 1H), 2.77-2.69 (m, 1H).

### EXAMPLE 15

### 1-(2,3-dideoxyl-3-methylidene-β-L-pentofuranosyl)-5-fluorocytosine (Compound 66)

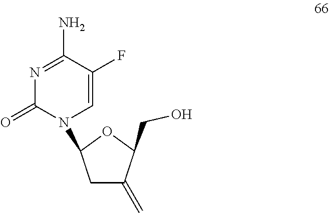

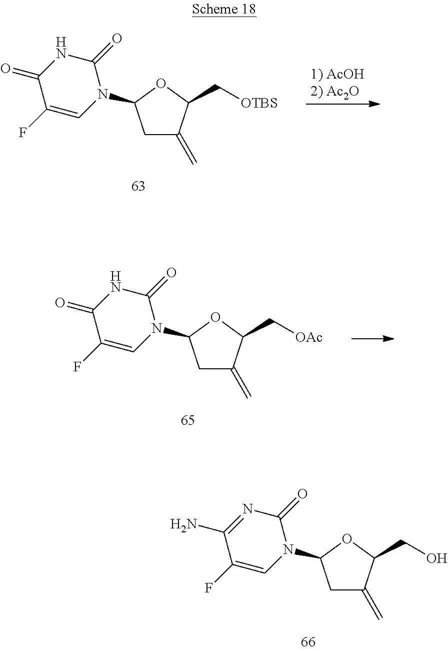

Compound 63 (27.5 mg 0.0771 mmol) was deprotected according to General procedure D. The residue was azeotropically dried with pyridine (2×2 mL) and then dissolved in dry pyridine (2 mL). To the resultant solution was added acetic anhydride (0.5 mL). The reaction mixture was stirred at RT for 24 h and then concentrated under reduced pressure. Flash chromatography (hexane:EtOAc 1:1) of the residue gave Compound 65 (16.6 mg) as a colorless solid.

To a solution of Compound 65 (16.6 mg 0.0584 mmol) in dry pyridine (1.1 mL) was added 4-chlorophenyl dichlorophosphate (47.5 μL, 0.292 mmol) at 0° C. After 10 min, 1,2,4-triazole (60.5 mg 0.876 mmol) was added and the temperature was allowed to reach RT. After 19 h, the reaction mixture was concentrated under reduced pressure and the residue was dissolved in H₂O. The aqueous phase was extracted twice with CH₂Cl₂. The combined organic extracts were dried (MgSO₄) and concentrated under reduced pressure. The residue was dissolved in NH₃ (6 mL, 0.5 M in dioxane) and the resultant solution was stirred at RT for 48 h and then concentrated. The residue was purified to give Compound 66 (3.3 mg) as a colorless solid.

¹H NMR (500 MHz, MeOH-d₄) δ=8.22 (d, J=6.8 Hz, 1H), 6.12 (tt, J=18.7, 9.3 Hz, 1H), 5.19 (dd, J=4.4, 2.2 Hz, 1H), 5.09 (dd, J=4.5, 2.2 Hz, 1H), 4.55 (m, 1H), 3.89 (dd, J=12.2, 2.9 Hz, 1H), 3.77 (dd, J=12.2, 3.9 Hz, 1H), 3.19-3.11 (m, 1H), 2.68-2.60 (m, 1H).

### EXAMPLE 16

### 9-(2,3-dideoxyl-3-methylidene-β-L-pentofuranosyl)adenine (Compound 71)

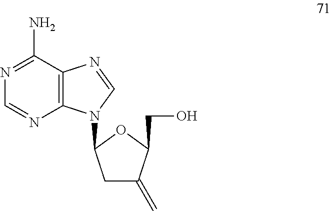

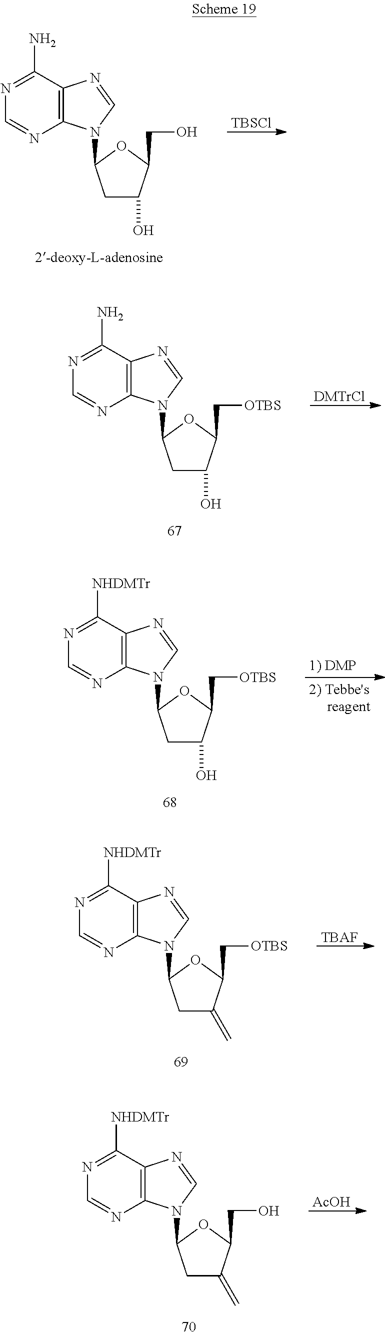

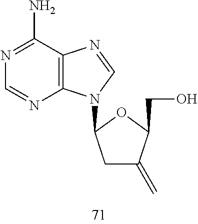

In a 50 mL round bottle 2′-deoxy-L-adenosine (900 mg 3.50 mmol) was azeotropically dried with pyridine (6×20 mL). Thereafter DMF (15 mL), imidazole (590 mg 8.00 mmol), and TBSCl (600 mg 4.00 mmol) were added. The resultant reaction mixture was stirred 6 h at room temperature after which MeOH (5 mL) was added and the mixture was stirred an additional 0.5 h. The solvents were then removed and the crude material was purified by flash chromatography (MeOH 0-5% in CH₂Cl₂) to give Compound 67 (1.0 g).

In a 50 mL round bottle Compound 67 (595 mg 1.62 mmol) was azeotropically dried with pyridine (3×20 mL). Thereafter pyridine (8 mL) and DMTrCl (610 mg 1.80 mmol) were added. The resultant reaction mixture was stirred 18 h at room temperature. More DMTrCl (300 mg 0.88 mmol) was added and the resultant mixture was stirred another 24 h. Then MeOH (5 mL) was added and the mixture was stirred an additional 10 min. The solvents was then removed by coevaporation with toluene and the crude material was purified by silica gel chromatography (0-3% MeOH in CH₂Cl₂, containing 0.1% pyridine) to give Compound 68 (526 mg).

In a 25 mL round bottle Dess-Martin periodinane (102 mg 0.24 mmol) was dried under vacuum for 30 min, thereafter CH₂Cl₂ (10 mL) and 2,6-di-tert-butylpyridine (191 mg 1.0 mmol) were added. Then Compound 68 (526 mg 0.789 mmol) was added and the reaction mixture was stirred 5 h at RT. The reaction mixture was diluted with EtOAc (10 mL) and was then poured into an aqueous solution of Na₂S₂O₃ (400 mg) in phosphate buffer (10 mL, pH 7.4). The resultant mixture was stirred vigorously for 2 min. The organic phase was separated and extracted with 1% NaHCO₃ aqueous solution (10 mL) for 5 seconds. The organic phase was separated, dried (MgSO₄) and concentrated under reduced pressure. The crude ketone was used immediately in the next step.

To a solution of the crude ketone in dry THF (5 mL) was added Tebbe's reagent (0.5 M in toluene, 0.60 mL, 0.30 mmol) dropwise at −78° C. The reaction mixture was stirred at −78° C. for 10 min and was then allowed to warm to room temperature and the reaction mixture was stirred for another 1 h. Then EtOAc (10 mL), MgSO₄.7 H₂O (1 g) and H₂O (1 mL) were added and the mixture was stirred for 10 min. Then MgSO₄ was added and the solids were filtered off. The crude reaction mixture was concentrated and purified by silica gel chromatography (0-1% MeOH in CH₂Cl₂, containing 0.1% pyridine) to give Compound 69 (4 mg).

To a solution of Compound 69 (4 mg 0.006 mmol) in THF (0.10 mL) was added TBAF (1M in THF, 0.030 mL) and the resultant reaction mixture was stirred at RT. After 20 min, NH₄Cl (0.1 mL) was added and the crude mixture was extracted with CH₂Cl₂ (3×0.3 mL), dried (MgSO₄) and concentrated under reduced pressure. Flash chromatography (hexane:EtOAc 1:1, containing 0.1% pyridine) of the residue gave Compound 70 (2.5 mg).

In a 2 mL vial Compound 70 (2.5 mg 0.0045 mmol) was dissolved in THF (0.20 mL), then AcOH (0.60 mL, 30% aq.) was added and the resultant reaction mixture was stirred at RT for 2.5 h. Thereafter the solvents were removed and the crude residue was dissolved in 2 mL H₂O and extracted with diethyl ether (2×2 mL). Activated charcoal was added in small portions to the aqueous phase until aqueous phase is no longer UV-active (spotted on TLC-plate). The charcoal suspension was loaded onto a flash column and the product was obtained by eluting the column with H₂O (50 mL) followed by H₂O:MeOH (50 mL, 1:1). Compound 71 (0.5 mg).

¹H NMR (500 MHz, MeOH-d₄) δ=8.32 (s, 1H), 8.19 (s, 1H), 6.33 (t, J=6.6 Hz, 1H), 5.29 (dd, J=4.3, 2.2 Hz, 1H), 5.17 (dd, J=4.4, 2.2 Hz, 1H), 4.66 (bs, 1H), 3.87 (dd, J=12.2, 2.9 Hz, 1H), 3.71 (dd, J=12.2, 4.1 Hz, 1H), 3.26-3.24 (m, 1H).

### EXAMPLE 17

### 9-(2,3-dideoxyl-3-methylidene-β-L-pentofuranosyl)guanine (Compound 75)

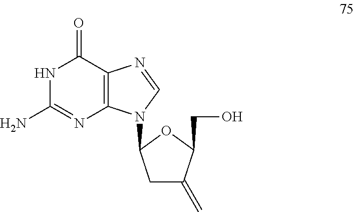

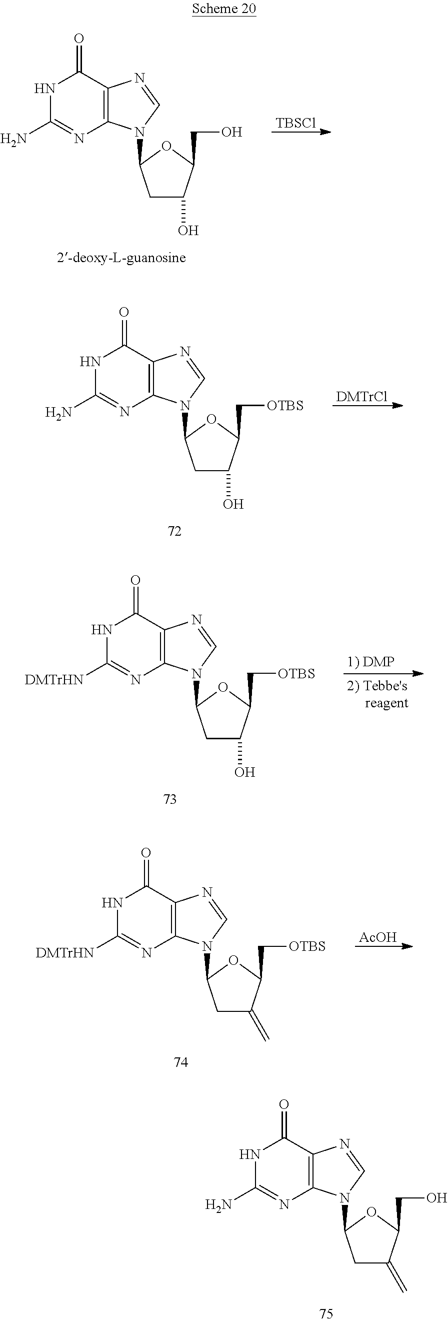

In a 50 mL flask 2′-deoxy-L-guanosine (1.00 g, 3.74 mmol) was azeotropically dried with pyridine (3×20 mL) and imidazole (660 mg 9.73 mmol) in DMF (20 mL) was added under N₂. Thereafter, TBSCl (732 mg 4.86 mmol) was added and the resultant mixture was stirred at 40° C. for 4 h. MeOH (2 mL) was added and the reaction mixture was stirred another 1 h at 40° C. The solvents were removed under reduced pressure and the crude material was purified by flash chromatography (0-10% MeOH in CH₂Cl₂) to give Compound 72 (1.23 g).

To a solution of Compound 72 (1.5 g, 3.93 mmol) in pyridine (20 mL) was added dimethoxytrityl chloride (2.66 g, 7.90 mmol) under N₂ and the resultant mixture was stirred at RT for 5 h. Then MeOH (1 mL) was added and the reaction mixture was stirred for 20 min. The solvents were then removed under reduced pressure and the crude material was purified by silica gel flash chromatography to give Compound 73 (1.44 g).

In a 10 mL flask Dess-Martin periodinane (614 mg 1.45 mmol) was dried in vacuo for 30 min, thereafter CH₂Cl₂ (12 mL) and t-BuOH (0.19 mL, 2.0 mmol) were added and the resultant mixture was stirred 10 min at 4° C. under N₂. Thereafter Compound 73 (762 mg 1.11 mmol) in CH₂Cl₂ (8 mL) was added. After 2 h the reaction mixture was diluted with CH₂Cl₂ (15 mL) and the mixture was transferred into an extraction funnel. Then 10% aq. NaHCO₃ (10 mL) containing Na₂S₂O₃.5H₂O (200 mg) was added and the mixture was shaken for 10 seconds. The organic phase was separated, dried (MgSO₄), and concentrated to give the crude ketone which was used immediately.

To a solution of the crude ketone in dry THF (6 mL) was added Tebbe's reagent (2.4 mL, 1.2 mmol, 0.5 M in toluene)drop wise at −78° C. under N₂. The reaction mixture was stirred for 10 min at −78° C. and was then allowed to warm to RT. After 2 h the reaction mixture was diluted with CH₂Cl₂ (20 mL) and then MgSO₄.7H₂O (10 g) and NaOH (1.0 M, 1 mL) were added, the mixture was stirred until the effervescence seized after which MgSO₄ was added. The mixture was filtered and concentrated under reduced pressure. Silica gel flash chromatography (0-5% MeOH in CH₂Cl₂) of the residue gave Compound 74 (159 mg) as a light brown solid.

To a solution of Compound 74 (80 mg 0.118 mmol) in THF (7 mL) was added

AcOH (28 mL, 30% aq.) and the resultant mixture was stirred at RT for 12 h. The solvents were removed in vacuo and traces of AcOH were removed by co-evaporation with H₂O. The crude residue was dissolved in H₂O (25 mL) and was then extracted with diethyl ether (2×20 mL). The water phase was concentrated to 5 mL, then activated charcoal was added in small portions to the aqueous phase until the aqueous phase was no longer UV-active (spotted on TLC-plate). The charcoal suspension was loaded onto a flash column and the product was obtained by eluting the column with H₂O (50 mL) followed by 50% MeOH in H₂O. Compound 75 (8 mg).

¹H NMR (500 MHz, MeOH-d₄) δ=7.93 (s, 1H), 6.16 (t, J=6.5 Hz, 1H), 5.26 (dd, J=4.6, 2.3 Hz, 1H), 5.15 (dd, J=4.4, 2.3 Hz, 2H), 3.83 (dd, J=12.1, 3.2 Hz, 1H), 3.70 (dd, J=12.1, 4.5 Hz, 1H), 3.24-3.21 (m, 1H), 3.19-3.16 (m, 1H).

### Biological Assays

### Cytotoxicity Assays

### Cytoxicity in HepG2 Cells:

HepG2 cells were seeded on 96-well plates at a density of 1×10⁴ cells/well in 100 μl DMEM supplemented with 10% FBS, 100 U/ml penicillin/streptomycin and 2 mM L-glutamine. After 20 hours of incubation at 37° C. with 5% CO₂, the medium was removed and replaced with fresh medium containing test compounds with the concentrations ranging 4.7-300 μM. The cells were incubated for 24 hours at 37° C. with 5% CO₂. The medium was removed and replaced with 100 μl/well MTT (Sigma) in HBSS (0.5 mg/ml). After the incubation for 2 hours on gentle shake at 37° C., 100 μl/well of MTT lysis buffer was added to the wells and plates were covered and left over night to dissolve formazan crystals. Absorbance was measured at 570-630 nm in ELISA reader. The cytotoxicity was calculated based on the cell viability in comparison with the control.

### Cytoxicity in MT-4 Cells:

MT-4 cells was added to 96-well microtiter plates in a volume of 50 μl (1×10⁵ cells/mL). The cell medium contains the test compounds with the concentrations ranging 0.01 μM-31 μM. The cells were incubated at 37° C. with 5% CO₂. At assay termination (6 days), 20-25 μL of MTS reagent is added per well and the microtiter plates are then incubated for 4-6 hrs at 37° C. with 5% CO₂. The plate was read spectrophotometrically to assess cell viability. The cytotoxicity was calculated based on the cell viability in comparison with the control.

### Anti-HIV Activity

MT-4 cells were used for analyzing compounds of the invention for their HIV inhibitory activities. On the day preceding the assay, the cells were split 1:2 to assure they were in an exponential growth phase at the time of infection. Total cell number and percent viability determinations were performed using a hemacytometer and trypan blue exclusion. Cell viability must be greater than 95% for the cells to be utilized in the assay. The cells were re-suspended at 1×10⁵ cells/mL in tissue culture medium and added to control or drug-containing 96-well microtiter plates in a volume of 50 μL.

The viruses used for this assay were HIV-1_{IIIB}. For each assay, a pre-titered aliquot of virus was removed from the freezer (−80° C.) and allowed to thaw slowly to room temperature in a biological safety cabinet. The virus was re-suspended and diluted into tissue culture medium such that the amount of virus added to each well in a volume of 50 μL was the amount determined to give between 85 to 95% cell killing at 6 days post-infection. The multiplicity of infection of these assays was approximately 0.01 and the volume added to the well of the microtiter plates was 50 μL.

At assay termination (6 days post-infection), 20-25 μL of MTS reagent 3-(4,5-dimethylthiazol-2-yl)-5-(3-carboxymethoxyphenyl)-2-(4-sulfophenyl)-2H-tetrazolium (MTS; CellTiter 96 Reagent, Promega) was added per well and the microtiter plates were incubated for 4-6 hrs at 37° C., 5% CO₂. Adhesive plate sealers were used in place of the lids, the sealed plate is inverted several times to mix the soluble formazan product. The plate was read spectrophotometrically at 490/650 nm with plate reader. Anti-HIV activity was calculated based on the Cytopathic Effect Reduction (%).

The HIV activity against resistant strains can be measured in a similar way using the cell culture assays infected by the resistant HIV strains.

### Anti-HBV Activity

Human hepatoma cells with HBV (HepG2.2.15 cells) were used for the analyzing compounds of the invention for their HBV inhibitory activities. HepG2.2.15 cells were plated in 96-well microtiter plates. Only the interior wells were utilized to reduce “edge effects” observed during cell culture; the exterior wells were filled with complete medium to help minimize sample evaporation. After 16-24 hours the confluent monolayer of HepG2.2.15 cells were washed and the medium was replaced with complete medium containing various concentrations of a test compound in triplicate (compounds tested at 6 concentrations). Lamivudine was used as the positive control, while media alone was added to cells as a negative control. Three days later the culture medium was replaced with fresh medium containing the appropriately diluted drug. Six days following the initial administration of the test compound, the cell culture supernatant was collected, treated with pronase and then used in a real-time quantitative TaqMan PCR assay. The PCR-amplified HBV DNA was detected in real-time by monitoring increases in fluorescence signals that result from the exonucleolytic degradation of a quenched fluorescent probe molecule that hybridized to the amplified HBV DNA. For each PCR amplification, a standard curve was simultaneously generated using dilutions of purified HBV DNA. Anti-HBV activity was calculated from the reduction in HBV DNA levels. CellTiter-96 kit (Promega) is employed to measure cell viability in the same assay to confirm that the inhibition was not due to cytotoxicity in HepG 2 cells.

Representative results of exemplified compounds of the invention from the assays are presented in Tables 1-4.

TABLE 1
Cytotoxicity in HepG2 cells

| | Cytotoxicity in |
|---|---|
| | HepG2 cells |
| Compounds | CC₅₀ (μM) |
| Compound 8 | >32 |
| Compound 10 | >300 |
| Compound 17 | >300 |
| Compound 26 | >300 |
| Compound 28 | >300 |
| Compound 37 | >300 |
| Compound 47 | >300 |
| Compound 57 | >300 |
| Compound 66 | >300 |
| Compound 71 | >300 |
| Compound 77 | >300 |

TABLE 2
Cytotoxicity in MT-4 cells

| | Cytotoxicity in MT-4 |
|---|---|
| Compounds | CC₅₀ (μM) |
| Compound 8 | >32 |
| Compound 10 | >32 |
| Compound 17 | >32 |
| Compound 26 | >32 |
| Compound 28 | >32 |
| Compound 37 | >32 |
| Compound 47 | >32 |
| Compound 75 | >32 |

TABLE 3
Anti-HBV activities in HepG2215 cells

| | Anti-HBV activity |
|---|---|
| Compounds | IC₅₀ (HepG 2215) |
| Compound 8 | A |
| Compound 10 | C |
| Compound 26 | C |
| Compound 66 | C |
| Compound 71 | C |

Category A: >32 μM;
Category B: 1-32 μM;
Category C: <1 μM

TABLE 4
Anti-HIV activities in MT-4 cells

| | Anti-HIV activity |
|---|---|
| Compounds | IC₅₀ (MT-4) |
| Compound 8 | A |
| Compound 10 | B |
| Compound 17 | A |
| Compound 26 | A |
| Compound 28 | A |
| Compound 37 | A |
| Compound 47 | A |
| Compound 75 | A |

Category A: >32 μM;
Category B: 1-32 μM;
Category C: <1 μM

1. A compound of general Formula (I) 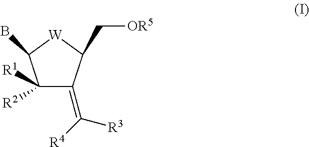
wherein
B is selected from A1 and A2; 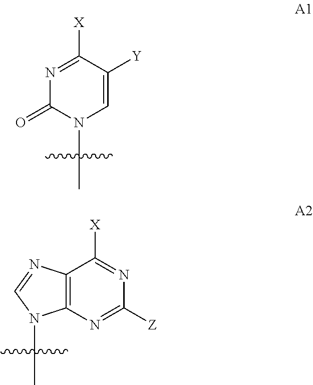
X is selected from H, OH, NH₂, halogen, (C₁-C₆alkyl)NH and (C₃-C₆cycloalkyl)NH;
Y is selected from H, halogen, C₂-C₆alkenyl and C₁-C₃alkyl;
Z is selected from H, halogen and NH₂;
W is selected from O, S and CH₂;
R¹ and R² are independently selected from H, F, OH, OCH₃ and CH₃;
R³ and R⁴ are independently selected from H, F and CH₃;
R⁵ is selected from H, phosphate, diphosphate and triphosphate;
or a pharmaceutically acceptable salt or prodrug thereof.

2. A compound according to claim 1, represented by general Formula (I) 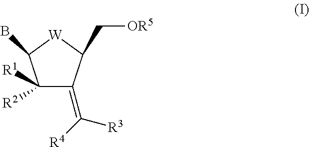
wherein
B is selected from A1 and A2; 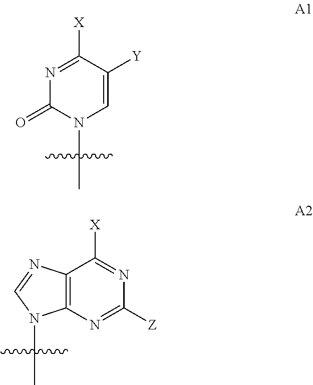
X is selected from H, OH, NH₂, halogen, (C₁-C₆alkyl)NH, and (C₃-C₆cycloalkyl)NH;
Y is selected from H, halogen, C₂-C₆alkenyl and C₁-C₃alkyl;
Z is selected from H, halogen and NH₂;
W is selected from O, S and CH₂;
R¹ and R² are independently selected from H, F, OH, OCH₃ and CH₃;
R³ and R⁴ are independently selected from H, F and CH₃;
R⁵ is selected from H, phosphate, diphosphate and triphosphate;
provided that when W is O; R¹ is H; and R² is OH, F or OCH₃, then R³ and R⁴ are not both F; or R³ and R⁴ are not both H; and
provided that when W is O; R² is H; and R¹ is OH, OCH₃ or F, then R³ and R⁴ are not both F; or R³ and R⁴ are not both H;
or a pharmaceutically acceptable salt or prodrug thereof.

3. A compound according to claim 1, wherein W is O.

4. A compound according to claim 1, wherein W is S or CH₂.

5. A compound according to claim 1, wherein R¹ and R² is H.

6. A compound according to claim 1, wherein R³ and R⁴ are independently selected from F and CH₃; provided that R³ and R⁴ are not both F.

7. A compound according to claim 1, wherein R³ is H; R⁴ is selected from F and CH₃.

8. A compound according to claim 1, wherein R⁴ is H; R³ is selected from F and CH₃.

9. A compound according to claim 1, wherein R¹ is CH₃.

10. A compound according to claim 1, wherein R² is CH₃.

11. A compound according to claim 1, wherein R¹, R², R³ and R⁴ is H.

12. A compound according to claim 1, wherein X is selected from H, OH and NH₂; Y is selected from H, F and CH₃; and Z is selected from H and NH₂.

13. A compound according to claim 1, wherein W is O; R² is OH or OCH₃; and R¹, R³ and R⁴ are H.

14. A compound according to claim 1, wherein W is O; R² is F; and R¹, R³ and R⁴ are H.

15. A compound according to claim 1, wherein W is O; R² is CH₃; and R¹, R³ and R⁴ are H.

16. A compound according to claim 1, wherein W is O; R¹ is F; and R², R³ and R⁴ are H.

17. A compound according to claim 1, wherein W is O; R¹ is OH or OCH₃; and R², R³ and R⁴ are H.

18. A compound according to claim 1, wherein B is A1; X is NH₂ or OH; Y is H, F or CH₃; W is O; R¹, R², R³ and R⁴ are H.

19. A compound according to claim 1, wherein B is A2; X is NH₂, OH or H; Z is H or NH₂; W is O; R¹, R², R³ and R⁴ are H.

20. A compound according to claim 1, wherein X is OH.

21. A compound according to of claim 1, wherein X is NH₂.

22. A compound according to claim 1, wherein Y is F.

23. A compound according to claim 1, wherein R⁵ is H.

24. A compound according to claim 1, wherein R⁵ is selected from phosphate, diphosphate and triphosphate.

25. A compound according to claim 1, selected from:
1-(2,3-dideoxy-3-methylidene-β-L-pentofuranosyl)cytosine;
1-(3-deoxy-3-methylidene-β-L-pentoribofuranosyl)cytosine;
1-(3-deoxy-3-methylidene-β-L-arabinopentofuranosyl)cytosine;
1-(2,3-dideoxy-3-methylidene-β-L-pentofuranosyl)thymine;
9-(2,3-dideoxy-3-methylidene-β-L-pentofuranosyl)guanine;
1-[2-deoxy-2-(S)-fluoro-3-deoxy-3-methylidene-β-L-pentofuranosyl]uracil;
1-[2-deoxy-2-(S)-fluoro-3-deoxy-3-methylidene-β-L-pentofuranosyl]cytosine;
1-[2-deoxy-2-(R)-fluoro-3-deoxy-3-methylidene-β-L-pentofuranosyl]uracil;
1-[2-deoxy-2-(R)-fluoro-3-deoxy-3-methylidene-β-L-pentofuranosyl]cytosine;
1-[(2S,3S,5R)-5-(Hydroxymethyl)-3-methyl-4-methylenetetrahydrofuran-2-yl]pyrimidine-2,4(1H,3H)-dione;
4-Amino-1-[(2S,3S,5R)-5-(hydroxymethyl)-3-methyl-4-methylenetetrahydrofuran-2-yl]pyrimidin-2(1H)-one;
1-(2,3-dideoxyl-3-methylidene-β-L-pentofuranosyl)-5-fluorouracil;
1-(2,3-dideoxyl-3-methylidene-β-L-pentofuranosyl)-5-fluorocytosine; and
9-(2,3-dideoxyl-3-methylidene-β-L-pentofuranosyl)adenine;
or a pharmaceutically acceptable salt or prodrug thereof.

26. A pharmaceutical composition for the treatment or prevention of a DNA virus infection and/or retroviral infection in a host comprising an effective amount of a compound according to claim 1.

27. A pharmaceutical composition for the treatment or prevention of HBV infections and/or HBV viruses which are resistant to one or more other anti-HBV drugs, comprising an effective amount of a compound according to claim 1.

28. A pharmaceutical composition for the treatment or prevention of HIV infections and/or HIV viruses which are resistant to one or more other anti-HIV drugs, comprising an effective amount of a compound according to claim 1.

29. The pharmaceutical composition according to claim 26, which further comprises one or more additional agents having antiviral effects.

30. A compound according to claim 1, for use in therapy.

31. A compound according to claim 1, for use in the treatment or prevention of a DNA virus infection and/or retroviral infection.

32. A compound according to claim 1, for use in the treatment or prevention of a HBV infection and/or a HBV virus which is resistant to one or more other anti-HBV drugs.

33. A compound according to claim 1, for use in the treatment or prevention of a HIV infection and/or a HIV virus which is resistant to one or more other anti-HIV drugs.

34. The compound for use according to claim 31, which use further comprises one or more additional agents having antiviral effects.

35. Use of a compound according to claim 1, in the manufacture of a medicament for treatment or prevention of a DNA virus infection and/or retroviral infection.

36. Use of a compound according to claim 1, in the manufacture of a medicament for treatment or prevention of a HBV virus infection; or a HBV virus, which is resistant to one or more other anti-HBV drugs.

37. Use of a compound according to claim 1, in the manufacture of a medicament for treatment or prevention of a HIV virus infection or a HIV virus, which is resistant to one or more other anti-HIV drugs.

38. The use according to claim 35, which use further comprises one or more additional agents having antiviral effects.

39. A method for the treatment or prevention of a DNA virus infection and/or retroviral infection in a subject in need thereof, comprising administering a therapeutically effective amount of a compound according to claim 1.

40. A method for the treatment or prevention of a HBV infection; or a HBV virus wherein said HBV virus is resistant to one or more other anti-HBV drugs, in a subject in need thereof, comprising administering a therapeutically effective amount of a compound according to claim 1.

41. A method for the treatment or prevention of a HIV infection; or a HIV virus wherein said HIV virus is resistant to one or more other anti-HIV drugs, in a subject in need thereof, comprising administering a therapeutically effective amount of a compound according to claim 1.

42. The method according to claim 39, which further comprises one or more additional agents having antiviral effects.
